(19) United States
(12) Patent Application Publication
(10) Pub. No.: US 2009/0042991 A1
(43) Pub. Date: Feb. 12, 2009

(54) Methods of increasing natural killer cell activity for therapy

(76) Inventors: James Barsoum, Lexington, MA (US); Zhenjian Du, Northborough, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/918,354

(22) PCT Filed: Apr. 13, 2006
(86) PCT No.: PCT/US2006/014320

§ 371 Date: Feb. 12, 2008

Related U.S. Application Data
(60) Provisional application No. 60/671,910, filed on Apr. 15, 2005.

(51) Int. Cl. A61K 31/15

(52) U.S. Cl. 514/599

(57) ABSTRACT

Methods of employing bis(thio-hydrazide amides) to increase NK cell activity in a subject in need thereof, e.g., a subject with an infection or an immunodeficiency, are provided such that the disorder is not cancer, a proliferative cell disorder, a non-infective heat shock protein 70 (Hsp70) responsive disorder, or a proteasome-inhibitor responsive disorder. Typically, a subject, e.g., a human, can be in need of increased NK cell activity has an immunodeficiency or is treated for an infection (e.g., a bacterial, viral, fungal, or parasite infection, or a combination thereof). The method includes administering to the subject an effective amount of a compound represented by Structural Formula I: Y is a covalent bond or an optionally substituted straight chained hydrocarbyl group, or, Y, taken together with both >C=Z groups to which it is bonded, is an optionally substituted aromatic group. R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring. R₇-R₈ are independently —H, an optionally substituted aliphatic group, or an optionally substituted aryl group. Z is O or S.

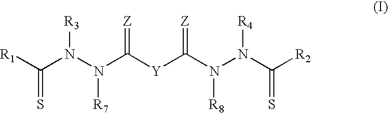

### RELATED APPLICATION

This application claims the benefit of U.S. Provisional Application No. 60/671,910, filed on Apr. 15, 2005. The entire teachings of the above application are incorporated herein by reference.

### BACKGROUND OF THE INVENTION

Natural killer (NK) cells, a type of white blood cell, are known to be an important component of the body's immune system. Because the defining function of NK cells is spontaneous cytotoxicity without prior immunization, NK cells can be the first line of defense in the immune system, and are believed to play a role in attacking cancer cells and infectious diseases. Many conditions, such as immunodeficiency diseases, aging, toxin exposure, endometriosis, and the like can leave subjects with lowered NK cell activity or dysfunctional NK cells.

For example, subjects can have decreased or deficient NK cell activity, in conditions such as chronic fatigue syndrome (chronic fatigue immune dysfunction syndrome) or Epstein-Barr virus, post viral fatigue syndrome, post-transplantation syndrome or host-graft disease, exposure to drugs such as anticancer agents or nitric oxide synthase inhibitors, natural aging, and various immunodeficiency conditions such as severe combined immunodeficiency, variable immunodeficiency syndrome, and the like. (Caligiuri M, Murray C, Buchwald D, Levine H, Cheney P, Peterson D, Komaroff A L, Ritz J. Phenotypic and functional deficiency of natural killer cells in patients with chronic fatigue syndrome. Journal of Immunology 1987; 139: 3306-13; Morrison L J A, Behan W H M, Behan P O. Changes in natural killer cell phenotype in patients with post-viral fatigue syndrome. Clinical and Experimental Immunology 1991; 83: 441-6; Klingemann, H G Relevance and Potential of Natural Killer Cells in Stem Cell Transplantation Biology of Blood and Marrow Transplantation 2000; 6:90-99; Ruggeri L, Capanni M, Mancusi A, Aversa F, Martelli M F, Velardi A. Natural killer cells as a therapeutic tool in mismatched transplantation. Best Pract Res Clin Haematol. 2004 September; 17(3):427-38; Cifone M G, Ulisse S, Santoni A. Natural killer cells and nitric oxide. Int Immunopharmacol. 2001 August; 1(8):1513-24; Plackett T P, Boehmer E D, Faunce D E, Kovacs E J. Aging and innate immune cells. J Leukoc Biol. 2004 August; 76(2):291-9. Epub 2004 Mar. 23; Alpdogan O, van den Brink M R. IL-7 and IL-15: therapeutic cytokines for immunodeficiency. Trends Immunol. 2005 January; 26(1):56-64; Heusel J W, Ballas Z K. Natural killer cells: emerging concepts in immunity to infection and implications for assessment of immunodeficiency. Curr Opin Pediatr. 2003 December; 15(6):586-93; Hacein-Bey-Abina S, Fischer A, Cavazzana-Calvo M. Gene therapy of X-linked severe combined immunodeficiency. Int J Hematol. 2002 November; 76(4):295-8; Baumert E, Schlesier M, Wolff-Vorbeck G, Peter H H. Alterations in lymphocyte subsets in variable immunodeficiency syndrome Immun Infekt. 1992 July; 20(3):73-5.)

NK cells are known to have activity against a wide range of infectious pathogens such as bacteria, viruses, fungi, protozoan parasites, combined infections, e.g., combined bacterial/viral infections, and the like. NK cells are believed to be particularly important in combating intracellular infections where the pathogens replicate in the subjects cells, e.g., a substantial fraction of viruses and many other pathogens that can form intracellular infections.

For example, a wide range of fungal infections are reported to be targeted by NK cells such as Cryptococcus neoformans, dermatophytes, e.g., Trichophyton rubrum, Candida albicans, Coccidioides immitis, Paracoccidioides brasiliensis, or the like (Hidore M R, Mislan T W, Murphy J W. Responses of murine natural killer cells to binding of the fungal target Cryptococcus neoformans Infect Immun. 1991 April; 59(4): 1489-99; Akiba H, Motoki Y, Satoh M, Iwatsuki K, Kaneko F; Recalcitrant trichophytic granuloma associated with NK-cell deficiency in a SLE patient treated with corticosteroid. Eur J Dermatol. 2001 January-February; 111(1):58-62; Mathews H L, Witek-Janusek L. Antifungal activity of interleukin-2-activated natural killer (NK1.1+) lymphocytes against Candida albicans. J Med Microbiol. 1998 November; 47(11):1007-14; Ampel N M, Bejarano G C, Galgiani J N. Killing of Coccidioides immitis by human peripheral blood mononuclear cells. Infect Immun. 1992 October; 60(10):4200-4; Jimenez B E, Murphy J W. In vitro effects of natural killer cells against Paracoccidioides brasiliensis yeast phase. Infect Immun. 1984 November; 46(2):552-8.)

Also targeted by NK cells are bacteria, especially intracellular bacteria, e.g., Mycobacterium tuberculosis, Mycobacterium avium, Listeria monocytogenes, many different viruses, such as human immunodeficiency virus, herpesviruses, hepatitis, and the like, and viral/bacterial co-infection (Esin S, Batoni G, Kallenius G, Gaines H, Campa M, Svenson S B, Andersson R, Wigzell H. Proliferation of distinct human T cell subsets in response to live, killed or soluble extracts of Mycobacterium tuberculosis and Myco. avium. Clin Exp Immunol. 1996 June; 104(3):419-25; Kaufmann S H. Immunity to intracellular bacteria. Annu Rev Immunol. 1993; 11:129-63; See D M, Khemka P, Sahl L, Bui T, Tilles J G. The role of natural killer cells in viral infections. Scand J Immunol. 1997 September; 46(3):217-24; Brenner B G, Dascal A, Margolese R G, Wainberg M A. Natural killer cell function in patients with acquired immunodeficiency syndrome and related diseases. J Leukoc Biol. 1989 July; 46(1):75-83; Kottilil S, Natural killer cells in HIV-1 infection: role of NK cell-mediated non-cytolytic mechanisms in pathogenesis of HIV-1 infection. Indian J Exp Biol. 2003 November; 41(11):1219-25; Herman R B, Koziel M J. Natural killer cells and hepatitis C: is losing inhibition the key to clearance? Clin Gastroenterol Hepatol. 2004 December; 2(12): 1061-3; Beadling C, Slifka M K. How do viral infections predispose patients to bacterial infections? Curr Opin Infect Dis. 2004 June; 17(3):185-91)

In addition, NK cells combat protozoal infections including toxoplasmosis, trypanosomiasis, leishmaniasis and malaria, especially intracellular infections (Korbel D S, Finney O C, Riley E M. Natural killer cells and innate immunity to protozoan pathogens. Int J Parasitol. 2004 December; 34(13-14): 1517-28; Ahmed J S, Mehlhorn H. Review: the cellular basis of the immunity to and immunopathogenesis of tropical theileriosis. Parasitol Res. 1999 July; 85(7):539-49; Osman M, Lausten S B, El-Sefi T, Boghdadi I, Rashed M Y, Jensen S L. Biliary parasites. Dig Surg. 1998; 15(4):287-96; Gazzinelli R T, Denkers E Y, Sher A. Host resistance to Toxoplasma gondii: model for studying the selective induction of cell-mediated immunity by intracellular parasites. Infect Agents Dis. 1993 June; 2(3): 139-49; Askonas B A, Bancroft G J. Interaction of African trypanosomes with the immune system. Philos Trans R Soc Lond B Biol Sci. 1984 Nov. 13; 307(1131):41-9; Allison A C, Eugui E M. The role of cell-mediated immune responses in resistance to malaria, with special reference to oxidant stress. Annu Rev Immunol. 1983; 1:361-92.)

Therefore, NK cells are known to be such an important component of the immune system. There is a continuing need in the art for effective treatments for increasing NK cell activity.

### SUMMARY OF THE INVENTION

It is now found that certain bis(thio-hydrazide) amides are surprisingly effective at maintaining or increasing NK cell activity. The methods disclosed herein demonstrate surprising biological activity by raising NK cell activity in humans (see Examples 3-6). Moreover, these surprising results were obtained in the presence of paclitaxel, which is known in the art to reduce NK cell activity.

Disclosed are methods employing bis(thio-hydrazide amides) to increase NK cell activity in a subject in need thereof, provided the disorder is not cancer, a proliferative cell disorder, a non-infective heat shock protein 70 (Hsp70) responsive disorder, or a proteasome-inhibitor responsive disorder.

Typically, a subject, e.g., a human, can be in need of increased NK cell activity has an immunodeficiency or is treated for an infection (e.g., a bacterial, viral, fungal, or parasite infection, or a combination thereof).

The method includes administering to the subject an effective amount of a compound represented by Structural Formula I:

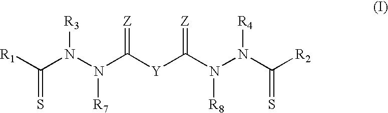

Y is a covalent bond or an optionally substituted straight chained hydrocarbyl group, or, Y, taken together with both >C=Z groups to which it is bonded, is an optionally substituted aromatic group.

R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring.

R₇-R₈ are independently —H, an optionally substituted aliphatic group, or an optionally substituted aryl group.

Z is O or S.

As used herein, the term “bis(thio-hydrazide amide)” also includes pharmaceutically acceptable salts and solvates of the compounds represented by Structural Formula I.

The methods described herein for increasing NK cell activity are believed to be effective for restoring or augmenting immune function, for example in subjects with immunodeficiency disorders, and to treating subjects (therapeutically or prophylactically) for infection, e.g., infections due to bacteria, fingi, viruses, parasites, or combinations thereof.

### BRIEF DESCRIPTION OF THE DRAWINGS

FIGS. 1A, 1B, and 1C are bar graphs showing the percent increase in Hsp70 plasma levels associated with administration of the Compound (1)/paclitaxel combination therapy at 1 hour (FIG. 1A), 5 hours (FIG. 1B), and 8 hours (FIG. 1C) after administration.

FIG. 2 is a Kaplan-Meier graph of time-to-progression (resumption of cancer growth) in studies of various combinations of platinum anticancer drugs and taxanes. Also shown is the disclosed combination of a bisthiohydrazide (Compound (1)), a taxane (paclitaxel) and also a platinum anticancer drug, carboplatin. The preliminary data shows that the disclosed method is superior to prior platin/taxane combinations alone.

### DETAILED DESCRIPTION OF THE INVENTION

A description of preferred embodiments of the invention follows.

The bis(thio-hydrazide amides) employed in the disclosed invention are represented by Structural Formula I and pharmaceutically acceptable salts and solvates of the compounds represented by Structural Formula I.

In one embodiment, Y in Structural Formula I is a covalent bond, —C(R₅R₆)—, —(CH₂CH₂)—, trans-(CH═CH)—, cis-(CH═CH)— or —(C≡C)— group, preferably —C(R₅R₆)—. R₁-R₄ are as described above for Structural Formula I. R₅ and R₆ are each independently —H, an aliphatic or substituted aliphatic group, or R₅ is —H and R₆ is an optionally substituted aryl group, or, R₅ and R₆, taken together, are an optionally substituted C₂-C₆ alkylene group. The pharmaceutically acceptable cation is as described in detail below.

In specific embodiments, Y taken together with both >C=Z groups to which it is bonded, is an optionally substituted aromatic group. In this instance, certain bis(thio-hydrazide amides) are represented by Structural Formula II:

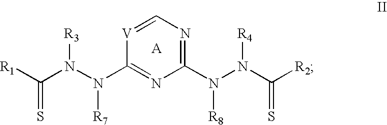

wherein Ring A is substituted or unsubstituted and V is —CH— or —N—. The other variables in Structural Formula II are as described herein for Structural Formula I or III.

In particular embodiments, the bis(thio-hydrazide amides) are represented by Structural Formula III:

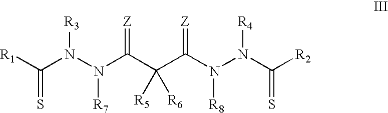

R₁-R₈ and the pharmaceutically acceptable cation are as described above for Structural Formula I.

In Structural Formulas I-III, R₁ and R₂ are the same or different and/or R₃ and R₄ are the same or different; preferably, R₁ and R₂ are the same and R₃ and R₄ are the same. In Structural Formulas I and III, Z is preferably O. Typically in Structural Formulas I and III, Z is O; R₁ and R₂ are the same; and R₃ and R₄ are the same. More preferably, Z is O; R₁ and R₂ are the same; R₃ and R₄ are the same, and R₇ and R₈ are the same.

In other embodiments, the bis(thio-hydrazide amides) are represented by Structural Formula III: R₁ and R₂ are each an optionally substituted aryl group, preferably an optionally substituted phenyl group; R₃ and R₄ are each an optionally substituted aliphatic group, preferably an alkyl group, more preferably, methyl or ethyl; and R₅ and R₆ are as described above, but R₅ is preferably —H and R₆ is preferably —H, an aliphatic or substituted aliphatic group.

Alternatively, R₁ and R₂ are each an optionally substituted aryl group; R₃ and R₄ are each an optionally substituted aliphatic group; R₅ is —H; and R₆ is —H, an aliphatic or substituted aliphatic group. Preferably, R₁ and R₂ are each an optionally substituted aryl group; R₃ and R₄ are each an alkyl group; and R₅ is —H and R₆ is —H or methyl. Even more preferably, R₁ and R₂ are each an optionally substituted phenyl group; R₃ and R₄ are each methyl or ethyl; and R₅ is —H and R₆ is —H or methyl. Suitable substituents for an aryl group represented by R₁ and R₂ and an aliphatic group represented by R₃, R₄ and R₆ are as described below for aryl and aliphatic groups.

In another embodiment, the bis(thio-hydrazide amides) are represented by Structural Formula III: R₁ and R₂ are each an optionally substituted aliphatic group, preferably a C₃-C₈ cycloalkyl group optionally substituted with at least one alkyl group, more preferably cyclopropyl or 1-methylcyclopropyl; R₃ and R₄ are as described above for Structural Formula I, preferably both an optionally substituted alkyl group; and R₅ and R₆ are as described above, but R₅ is preferably —H and R₆ is preferably —H, an aliphatic or substituted aliphatic group, more preferably —H or methyl.

Alternatively, the bis(thio-hydrazide amides) are represented by Structural Formula III: R₁ and R₂ are each an optionally substituted aliphatic group; R₃ and R₄ are as described above for Structural Formula I, preferably both an optionally substituted alkyl group; and R₅ is —H and R₆ is —H or an optionally substituted aliphatic group. Preferably, R₁ and R₂ are both a C₃-C₈ cycloalkyl group optionally substituted with at least one alkyl group; R₃ and R₄ are both as described above for Structural Formula I, preferably an alkyl group; and R₅ is —H and R₆ is —H or an aliphatic or substituted aliphatic group. More preferably, R₁ and R₂ are both a C₃-C₈ cycloalkyl group optionally substituted with at least one alkyl group; R₃ and R₄ are both an alkyl group; and R₅ is —H and R₆ is —H or methyl. Even more preferably, R₁ and R₂ are both cyclopropyl or 1-methylcyclopropyl; R₃ and R₄ are both an alkyl group, preferably methyl or ethyl; and R₅ is —H and R₆ is —H or methyl.

In specific embodiments, the bis(thio-hydrazide amides) are represented by Structural Formula IV:

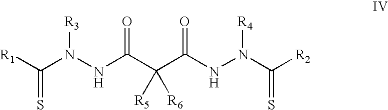

wherein: R₁ and R₂ are both phenyl, R₃ and R₄ are both methyl, and R₅ and R₆ are both —H; R₁ and R₂ are both phenyl, R₃ and R₄ are both ethyl, and R₅ and R₆ are both —H; R₁ and R₂ are both 4-cyanophenyl, R₃ and R₄ are both methyl, R₅ is methyl, and R₆ is —H; R₁ and R₂ are both 4-methoxyphenyl, R₃ and R₄ are both methyl, and R₅ and R₆ are both —H; R₁ and R₂ are both phenyl, R₃ and R₄ are both methyl, R₅ is methyl, and R₆ is —H; R₁ and R₂ are both phenyl, R₃ and R₄ are both ethyl, R₅ is methyl, and R₆ is —H; R₁ and R₂ are both 4-cyanophenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both ₃-cyanophenyl, R₃ and R₄ are both methyl, and R₅ and R₆ are both —H; R₁ and R₂ are both ₃-fluorophenyl, R₃ and R₄ are both methyl, and R₅ and R₆ are both —H; R₁ and R₂ are both 4-chlorophenyl, R₃ and R₄ are both methyl, R₅ is methyl, and R₆ is —H; R₁ and R₂ are both 2-dimethoxyphenyl, R₃ and R₄ are both methyl, and R₅ and R₆ are both —H; R₁ and R₂ are both 3-methoxyphenyl, R₃ and R₄ are both methyl, and R₅ and R₆ are both —H; R₁ and R₂ are both 2,3-dimethoxyphenyl, R₃ and R₄ are both methyl, and R₅ and R₆ are both —H; R₁ and R₂ are both 2,3-dimethoxyphenyl, R₃ and R₄ are both methyl, R₅ is methyl, and R₆ is —H; R₁ and R₂ are both 2,5-difluorophenyl, R₃ and R₄ are both methyl, and R₅ and R₆ are both —H; R₁ and R₂ are both 2,5-difluorophenyl, R₃ and R₄ are both methyl, R₅ is methyl, and R₆ is —H; R₁ and R₂ are both 2,5-dichlorophenyl, R₃ and R₄ are both methyl, and R₅ and R₆ are both —H; R₁ and R₂ are both 2,5-dimethylphenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both phenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both cyclopropyl, R₃ and R₄ are both methyl, and R₅ and R₆ are both —H; R₁ and R₂ are both cyclopropyl, R₃ and R₄ are both ethyl, and R₅ and R₆ are both —H; R₁ and R₂ are both cyclopropyl, R₃ and R₄ are both methyl, R₅ is methyl, and R₆ is —H; R₁ and R₂ are both 1-methylcyclopropyl, R₃ and R₄ are both methyl, and R₅ and R₆ are both —H; R₁ and R₂ are both 1-methylcyclopropyl, R₃ and R₄ are both methyl, R₅ is methyl and R₆ is —H; R₁ and R₂ are both 1-methylcyclopropyl, R₃ and R₄ are both methyl, R₅ is ethyl, and R₆ is —H; R₁ and R₂ are both 1-methylcyclopropyl, R₃ and R₄ are both methyl, R₅ is n-propyl, and R₆ is —H; R₁ and R₂ are both 1-methylcyclopropyl, R₃ and R₄ are both methyl, and R₅ and R₆ are both methyl; R₁ and R₂ are both 1-methylcyclopropyl, R₃ and R₄ are both ethyl, and R₅ and R₆ are both —H; R₁ and R₂ are both 1-methylcyclopropyl, R₃ is methyl, R₄ is ethyl, and R₅ and R₆ are both —H; R₁ and R₂ are both 2-methylcyclopropyl, R₃ and R₄ are both methyl, and R₅ and R₆ are both —H; R₁ and R₂ are both 2-phenylcyclopropyl, R₃ and R₄ are both methyl, and R₅ and R₆ are both —H; R₁ and R₂ are both 1-phenylcyclopropyl, R₃ and R₄ are both methyl, and R₅ and R₆ are both —H; R₁ and R₂ are both cyclobutyl, R₃ and R₄ are both methyl, and R₅ and R₆ are both —H; R₁ and R₂ are both cyclopentyl, R₃ and R₄ are both methyl, and R₅ and R₆ are both —H; R₁ and R₂ are both cyclohexyl, R₃ and R₄ are both methyl, and R₅ and R₆ are both —H; R₁ and R₂ are both cyclohexyl, R₃ and R₄ are both phenyl, and R₅ and R₆ are both —H; R₁ and R₂ are both methyl, R₃ and R₄ are both methyl, and R₅ and R₆ are both —H; R₁ and R₂ are both methyl, R₃ and R₄ are both t-butyl, and R₅ and R₆ are both —H; R₁ and R₂ are both methyl, R₃ and R₄ are both phenyl, and R₅ and R₆ are both —H; R₁ and R₂ are both t-butyl, R₃ and R₄ are both methyl, and R₅ and R₆ are both —H; R₁ and R₂ are ethyl, R₃ and R₄ are both methyl, and R₅ and R₆ are both —H; or R₁ and R₂ are both n-propyl, R₃ and R₄ are both methyl, and R₅ and R₆ are both —H.

In specific embodiments, the bis(thio-hydrazide amides) are represented by Structural Formula V:

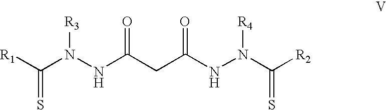

wherein: R₁ and R₂ are both phenyl, and R₃ and R₄ are both o-CH₃-phenyl; R₁ and R₂ are both o-CH₃C(O)O-phenyl, and R₃ and R₄ are phenyl; R₁ and R₂ are both phenyl, and R₃ and R₄ are both methyl; R₁ and R₂ are both phenyl, and R₃ and R₄ are both ethyl; R₁ and R₂ are both phenyl, and R₃ and R₄ are both n-propyl; R₁ and R₂ are both p-cyanophenyl, and R₃ and R₄ are both methyl; R₁ and R₂ are both p-nitro phenyl, and R₃ and R₄ are both methyl; R₁ and R₂ are both 2,5-dimethoxyphenyl, and R₃ and R₄ are both methyl; R₁ and R₂ are both phenyl, and R₃ and R₄ are both n-butyl; R₁ and R₂ are both p-chlorophenyl, and R₃ and R₄ are both methyl; R₁ and R₂ are both 3-nitrophenyl, and R₃ and R₄ are both methyl; R₁ and R₂ are both 3-cyanophenyl, and R₃ and R₄ are both methyl; R₁ and R₂ are both 3-fluorophenyl, and R₃ and R₄ are both methyl; R₁ and R₂ are both 2-furanyl, and R₃ and R₄ are both phenyl; R₁ and R₂ are both 2-methoxyphenyl, and R₃ and R₄ are both methyl; R₁ and R₂ are both 3-methoxyphenyl, and R₃ and R₄ are both methyl; R₁ and R₂ are both 2,3-dimethoxyphenyl, and R₃ and R₄ are both methyl; R₁ and R₂ are both 2-methoxy-5-chlorophenyl, and R₃ and R₄ are both ethyl; R₁ and R₂ are both 2,5-difluorophenyl, and R₃ and R₄ are both methyl; R₁ and R₂ are both 2,5-dichlorophenyl, and R₃ and R₄ are both methyl; R₁ and R₂ are both 2,5-dimethylphenyl, and R₃ and R₄ are both methyl; R₁ and R₂ are both 2-methoxy-5-chlorophenyl, and R₃ and R₄ are both methyl; R₁ and R₂ are both 3,6-dimethoxyphenyl, and R₃ and R₄ are both methyl; R₁ and R₂ are both phenyl, and R₃ and R₄ are both 2-ethylphenyl; R₁ and R₂ are both 2-methyl-5-pyridyl, and R₃ and R₄ are both methyl; or R₁ is phenyl; R₂ is 2,5-dimethoxyphenyl, and R₃ and R₄ are both methyl; R₁ and R₂ are both methyl, and R₃ and R₄ are both p-CF₃-phenyl; R₁ and R₂ are both methyl, and R₃ and R₄ are both o-CH₃-phenyl; R₁ and R₂ are both —(CH₂)₃COOH; and R₃ and R₄ are both phenyl; R₁ and R₂ are both represented by the following structural formula:

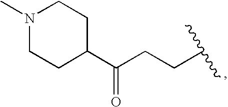

and R₃ and R₄ are both phenyl; R₁ and R₂ are both n-butyl, and R₃ and R₄ are both phenyl; R₁ and R₂ are both n-pentyl, R₃ and R₄ are both phenyl; R₁ and R₂ are both methyl, and R₃ and R₄ are both 2-pyridyl; R₁ and R₂ are both cyclohexyl, and R₃ and R₄ are both phenyl; R₁ and R₂ are both methyl, and R₃ and R₄ are both 2-ethylphenyl; R₁ and R₂ are both methyl, and R₃ and R₄ are both 2,6-dichlorophenyl; R₁-R₄ are all methyl; R₁ and R₂ are both methyl, and R₃ and R₄ are both t-butyl; R₁ and R₂ are both ethyl, and R₃ and R₄ are both methyl; R₁ and R₂ are both t-butyl, and R₃ and R₄ are both methyl; R₁ and R₂ are both cyclopropyl, and R₃ and R₄ are both methyl; R₁ and R₂ are both cyclopropyl, and R₃ and R₄ are both ethyl; R₁ and R₂ are both 1-methylcyclopropyl, and R₃ and R₄ are both methyl; R₁ and R₂ are both 2-methylcyclopropyl, and R₃ and R₄ are both methyl; R₁ and R₂ are both 1-phenylcyclopropyl, and R₃ and R₄ are both methyl; R₁ and R₂ are both 2-phenylcyclopropyl, and R₃ and R₄ are both methyl; R₁ and R₂ are both cyclobutyl, and R₃ and R₄ are both methyl; R₁ and R₂ are both cyclopentyl, and R₃ and R₄ are both methyl; R₁ is cyclopropyl, R₂ is phenyl, and R₃ and R₄ are both methyl.

Preferred examples of bis(thio-hydrazide amides) include Compounds (1)-(18) and pharmaceutically acceptable salts and solvates thereof:

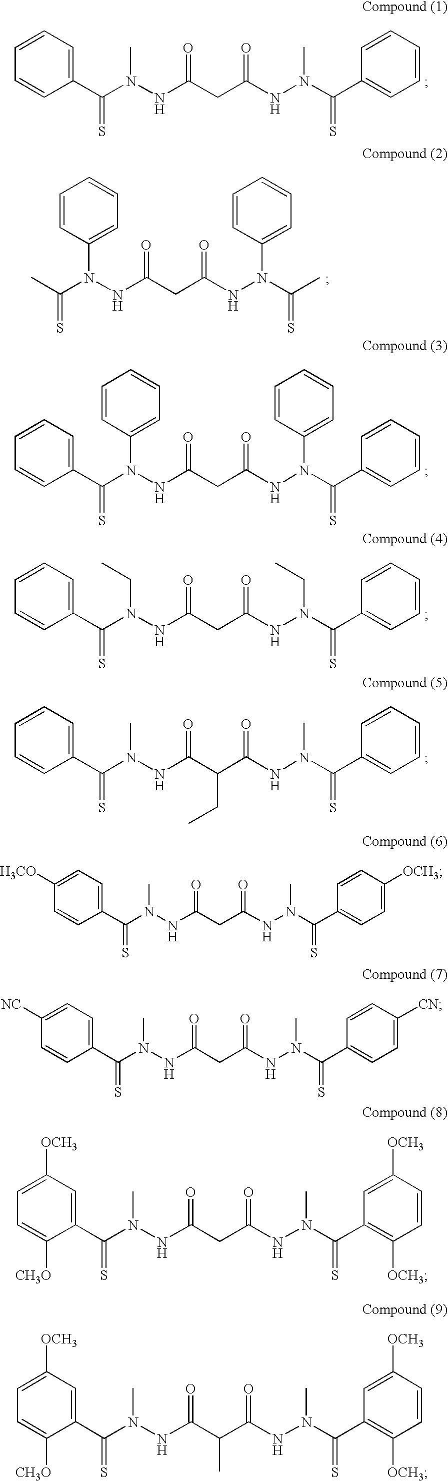

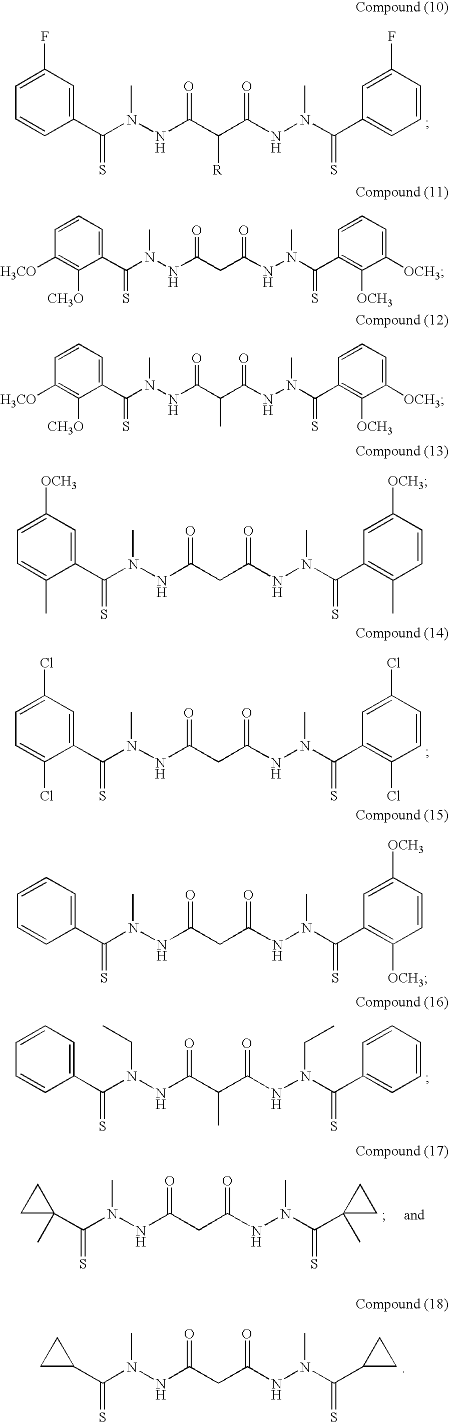

Particular examples of bis(thio-hydrazide amides) include Compounds (1), (17), and (18) and pharmaceutically acceptable salts and solvates thereof.

A “straight chained hydrocarbyl group” is an alkylene group, i.e., —(CH₂)_{y}—, with one, or more (preferably one) internal methylene groups optionally replaced with a linkage group. y is a positive integer (e.g., between 1 and 10), preferably between 1 and 6 and more preferably 1 or 2. A “linkage group” refers to a functional group which replaces a methylene in a straight chained hydrocarbyl. Examples of suitable linkage groups include a ketone (—C(O)—), alkene, alkyne, phenylene, ether (—O—), thioether (—S—), or amine (—N(R^{a})—), wherein R^{a} is defined below. A preferred linkage group is —C(R₅R₆)—, wherein R₅ and R₆ are defined above. Suitable substituents for an alkylene group and a hydrocarbyl group are those which do not substantially interfere with the activity of the bis(thio-hydrazide) amides. R₅ and R₆ are preferred substituents for an alkylene or hydrocarbyl group represented by Y.

An aliphatic group is a straight chained, branched or cyclic non-aromatic hydrocarbon which is completely saturated or which contains one or more units of unsaturation. Typically, a straight chained or branched aliphatic group has from 1 to about 20 carbon atoms, preferably from 1 to about 10, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. A C₁-C₂₀ straight chained or branched alkyl group or a C₃-C₈ cyclic alkyl group is also referred to as a “lower alkyl” group.

The term “aromatic group” may be used interchangeably with “aryl,” “aryl ring,” “aromatic ring,” “aryl group” and “aromatic group.” Aromatic groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazole, oxazolyl, and tetrazole. The term “heteroaryl group” may be used interchangeably with “heteroaryl,” “heteroaryl ring,” “heteroaromatic ring” and “heteroaromatic group.” The term “heteroaryl,” as used herein, means a mono- or multi-cyclic aromatic heterocycle which comprise at least one heteroatom such as nitrogen, sulfur and oxygen, but may include 1, 2, 3 or 4 heteroatoms per ring. Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

The term “arylene” refers to an aryl group which is connected to the remainder of the molecule by two other bonds. By way of example, the structure of a 1,4-phenylene group is shown below:

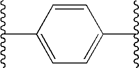

Substituents for an arylene group are as described below for an aryl group.

Non-aromatic heterocyclic rings are non-aromatic rings which include one or more heteroatoms such as nitrogen, oxygen or sulfur in the ring. The ring can be five, six, seven or eight-membered. Examples include tetrahydrofuranyl, tetrahydrothiophenyl, morpholino, thiomorpholino, pyrrolidinyl, piperazinyl, piperidinyl, and thiazolidinyl.

Suitable substituents on an aliphatic group (including an alkylene group), non-aromatic heterocyclic group, benzylic or aryl group (carbocyclic and heteroaryl) are those which do not substantially interfere with the activity of the bis(thio-hydrazide) amides. A substituent substantially interferes with activity when the activity is reduced by more than about 50% in a compound with the substituent compared with a compound without the substituent. Examples of suitable substituents include —R^{a}, —OH, —Br, —Cl, —I, —F, —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NRC)—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{b}, —CH═CHR^{a}, —CH═CR^{a}R^{b}, —CR^{c}═CR^{a}R^{b}, —CR^{c}═CHR^{a}, —CRC═CR^{a}R^{b}, —CCR^{a}, —SH, —SR^{a}, —S(O)R^{a}, —S(O)₂R^{a}. R^{a}-R^{d} are each independently an alkyl group, aromatic group, non-aromatic heterocyclic group or —N(R^{a}R^{b}), taken together, form an optionally substituted non-aromatic heterocyclic group. The alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}.

R^{#} is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R₊, —C(O)R⁺, —C(O)N(R)₂, —OC(O)R⁺, —OC(O)N(R⁺)₂, —S(O)₂R₊, —SO₂N(R⁺)₂, —S(O)R⁺, —NHSO₂N(R⁺)₂, —NHSO₂R⁺, —C(═S)N(R⁺)₂, or —C(—NH)—N(R⁺)₂.

R⁺ is —H, a C₁-C₄ alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine. Optionally, the group —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

Preferred substituents for a phenyl group, including phenyl groups represented by R₁-R₄, include C₁-C₄ alkyl, C₁-C₄ alkoxy, C₁-C₄ haloalkyl, C₁-C₄ haloalkoxy, phenyl, benzyl, pyridyl, —OH, —NH₂, —F, —Cl, —Br, —F, —NO₂ or —CN.

Preferred substituents for an aliphatic group, including aliphatic groups represented by R₁-R₄, include C₁-C₄ alkyl, C₁-C₄ alkoxy, C₁-C₄ haloalkyl, C₁-C₄ haloalkoxy, phenyl, benzyl, pyridyl, —OH, —NH₂, —F, —Cl, —Br, —I, —NO₂ or —CN.

Preferred substituents for a cycloalkyl group, including cycloalkyl groups represented by R₁ and R₂, are alkyl groups, such as a methyl or ethyl groups.

Also included in the present invention are pharmaceutically acceptable salts of the bis(thio-hydrazide) amides employed herein. These compounds can have one or more sufficiently acidic protons that can react with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, allyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of bis(thio-hydrazide) amides employed herein (e.g., those represented by Structural Formulas I-VI, Compounds 1-18,) are those formed by the reaction of the compound with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). Divalent salts of the bis(thio-hydrazide amides) are preferred. “Pharmaceutically acceptable” means that the cation is suitable for administration to a subject. Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C₂H₅OH)⁺ or N(CH₃)₃(C₂H₅OH)⁺, and more typically, the salt is a disodium or dipotassium salt, preferably the disodium salt.

Bis(thio-hydrazide) amides employed herein having a sufficiently basic group, such as an amine can react with an organic or inorganic acid to form an acid addition salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Particular salts of the bis(thio-hydrazide amide) compounds described herein can be prepared according to methods described in copending, co-owned Patent Application Ser. No. 60/582,596, filed Jun. 23, 2004.

The neutral bis(thio-hydrazide) amides can be prepared according to methods described in U.S. Pat. Nos. 6,800,660, and 6,762,204, both entitled “Synthesis of Taxol Enhancers” and also according to methods described in the co-pending and co-owned U.S. patent application Ser. Nos. 10/345,885 filed Jan. 15, 2003, and 10/758,589, Jan. 15, 2004. The entire teachings of each document referred to in this application is expressly incorporated herein by reference.

It will also be understood that certain compounds employed in the invention may be obtained as different stereoisomers (e.g., diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

A “subject” includes mammals, e.g., humans, companion animals (e.g., dogs, cats, birds, aquarium fish, reptiles, and the like), farm animals (e.g., cows, sheep, pigs, horses, fowl, farm-raised fish and the like) and laboratory animals (e.g., rats, mice, guinea pigs, birds, aquarium fish, reptiles, and the like). Alternatively, the subject is a warm-blooded animal. More preferably, the subject is a mammal. Most preferably, the subject is human.

A subject in need of treatment is in need of immune system augmentation because of infection or the possibility thereof. In some embodiments, such a subject can have an infection (or has been exposed to an infectious environment where pathogens are present, e.g., in a hospital) the symptoms of which may be alleviated by the methods disclosed herein. For example, a subject in need of treatment can have an infection (bacterial, viral, fungal, or parasitical (protozoal) for which the disclosed methods of activating NK cells can be a treatment.

In some embodiments, a subject in need of treatment is in need of immune system augmentation because the subject has an immunodeficiency. Such a subject is in need of or can benefit from prophylactic therapy, for example, a subject that has incomplete, damaged or otherwise compromised defenses against infection, or is subject to an infective environment, or the like. For example, a subject can be in an infectious environment where pathogens are present, e.g., in a hospital; can have an open wound or burn injury; can have an inherited or acquired immune deficiency (e.g., severe combined immunodeficiency or “bubble boy” syndrome, variable immunodeficiency syndrome acquired immune deficiency syndrome (AIDS), or the like); can have a depressed immune system due to physical condition, age, toxin exposure, drug effect (immunosuppressants, e.g., in a transplant recipient) or side effect (e.g., due to an anticancer agent); or the like.

In some embodiments, NK activity can be increased in subjects that have decreased or deficient NK cell activity, in conditions such as chronic fatigue syndrome (chronic fatigue immune dysfunction syndrome) or Epstein-Barr virus infection, post viral fatigue syndrome, post-transplantation syndrome (especially allogeneic transplants) or host-graft disease, exposure to drugs such as anticancer agents or nitric oxide synthase inhibitors, natural aging, and various immunodeficient conditions such as severe combined immunodeficiency, variable immunodeficiency syndrome, and the like.

In some embodiments, the subject is in need of treatment for bacteremia. Bacteremia is the condition of bacterial infection in the bloodstream. Septic shock includes serious localized or bacteremic infection accompanied by systemic inflammation, in other words sepsis with hypoperfusion and hypotension refractory to fluid therapy. Sepsis, or systemic inflammatory response syndrome, includes various severe conditions such as infections, pancreatitis, burns, trauma) that can cause acute inflammation. Septic shock is typically related to infections by gram-negative organisms, staphylococci, or meningococci. Septic shock can be characterized by acute circulatory failure, typically with hypotension, and multiorgan failure.

In some embodiments, the methods do not include sepsis.

Transient bacteremia can be caused by surgical or trauma wounds. Gram-negative bacteremia can be intermittent and opportunistic; although it may have no effect on a healthy person, it may be seriously important in immunocompromised patients with debilitating underlying diseases, after chemotherapy, and in settings of malnutrition. The infection can typically be in the lungs, in the GU or GI tract, or in soft tissues, e.g., skin in patients with decubitus ulcer, oral ulcers in patients at risk, and patients with valvular heart disease, prosthetic heart valves, or other implanted prostheses.

Typically, gram-negative bacteremia can manifest in chronically ill and immunocompromised patients. Also in such patients, bloodstream infections can be caused by aerobic bacilli, anaerobes, and fungi. Bacteroides can lead to abdominal and pelvic infective complications, especially in females. Transient or sustained bacteremia can typically result in metastatic infection of the meninges or serous cavities, such as the pericardium or larger joints. Enterococcus, staphylococcus, or fungus can lead to endocarditis, but is less common with gram-negative bacteremia. Staphylococcal bacteremia can be typical of IV drug users, and can be a typical cause of gram-positive bacterial endocarditis.

The incidence of systemic fungal infections has undergone a significant increase, particularly in humans, due in part to increases in the number of subjects with compromised immune systems, for example, the elderly, AIDS patients, patients undergoing chemotherapy, burn patients, patients with diabetic ketoacidosis, and transplant patients on immunosuppressive drugs. A study found that about 40% of deaths from infections acquired during hospitalization were due to mycoses; see Sternberg et. al, Science, Vol. 266, (1994), pp. 1632-1634, the entire teachings of which are incorporated herein by reference.

In various embodiments, the subject can be treated for a fungal infection from a pathogenic dermatophyte, a pathogenic filamentous fungus, and/or a pathogenic non-filamentous fungus, e.g., a yeast, or the like. Pathogenic dermatophytes can include, e.g., species of the genera Trichophyton, Tinea, Microsporum, Epidermophyton, or the like. Pathogenic filamentous fungus can include, e.g., species of genera such as Aspergillus, Histoplasma, Cryptococcus, Microsporum, or the like. Pathogenic non-filamentous fungus, e.g., yeasts, can include, for example, species of the genera Candida, Malassezia, Trichosporon, Rhodotorula, Torulopsis, Blastomyces, Paracoccidioides, Coccidioides, or the like. In various embodiments, the subject can be treated for a fungal infection from a species of the genera Aspergillus or Trichophyton. Species of Trichophyton can include, for example, Trichophyton mentagrophytes, Trichophyton rubrum, Trichophyton schoenleinii, Trichophyton tonsurans, Trichophyton verrucosum, and Trichophyton violaceum. Species of Aspergillus can include, for example, Aspergillus fumigatus, Aspergillus flavus, Aspergillus niger, Aspergillus amstelodami, Aspergillus candidus, Aspergillus carneus, Aspergillus nidulans, A oryzae, Aspergillus restrictus, Aspergillus sydowi, Aspergillus terreus, Aspergillus ustus, Aspergillus versicolor; Aspergillus caesiellus, Aspergillus clavatus, Aspergillus avenaceus, and Aspergillus deflectus. In some embodiments, the subject can be treated for a fungal infection from a pathogenic dermatophyte, e.g., Trichophyton (e.g., Trichophyton rubrum), Tinea, Microsporum, or Epidermophyton; or Cryptococcus (e.g., Cryptococcus neoformans) Candida (e.g., Candida albicans), Paracoccidioides (e.g., Paracoccidioides brasiliensis), or Coccidioides (e.g., Coccidioides immitis). In particular embodiments, the subject can be treated for a fungal infection from Trichophyton rubrum, Cryptococcus neoformans, Candida albicans, Paracoccidioides brasiliensis, or Coccidioides immitis.

Thus, in various embodiments, a subject can have an infection caused by a fungus selected from the genera Trichophyton, Tinea, Microsporum, Epidermophyton, Aspergillus, Histoplasma, Cryptococcus, Microsporum, Candida, Malassezia, Trichosporon, Rhodotorula, Torulopsis, Blastomyces, Paracoccidioides, and Coccidioides. In some embodiments, the subject can have an infection caused by a fungus selected from the genera Trichophyton, Tinea, Microsporum, Epidermophyton; Cryptococcus, Candida, Paracoccidioides, and Coccidioides. In certain embodiments, the subject can have an infection caused by a fungus selected from Trichophyton rubrum, Cryptococcus neoformans, Candida albicans, Paracoccidioides brasiliensis, and Coccidioides immitis.

In various embodiments, the subject can be treated for a bacterial infection caused by a bacteria of a genus selected from Allochromatium, Acinetobacter, Bacillus, Campylobacter, Chlamydia, Chlamydophila, Clostridium, Citrobacter, Escherichia, Enterobacter, Enterococcus, Francisella, Haemophilus, Helicobacter, Klebsiella, Listeria, Moraxella, Mycobacterium, Micrococcus, Neisseria, Proteus, Pseudomonas, Salmonella, Serratia, Shigella, Stenotrophomonas, Staphyloccocus, Streptococcus, Synechococcus, Vibrio, and Yersina; or anerobic bacterial genera such as Peptostreptococci, Porphyromonas, Actinomyces, Clostridium, Bacteroides, Prevotella, Anaerobiospirillum, Fusobacterium, and Bilophila. In some embodiments, the subject can be treated for a bacterial infection from Allochromatium vinosum, Acinetobacter baumanii, Bacillus anthracis, Campylobacter jejuni, Chlamydia trachomatis, Chlamydia pneumoniae, Clostridium spp., Citrobacter spp., Escherichia coli, Enterobacter spp., Enterococcus faecalis, Enterococcus faecium, Francisella tularensis, Haemophilus influenzae, Helicobacter pylori, Klebsiella spp., Listeria monocytogenes, Moraxella catarrhalis, Mycobacterium tuberculosis, Neisseria meningitidis, Neisseria gonorrhoeae, Proteus mirabilis, Proteus vulgaris, Pseudomonas aeruginosa, Salmonella spp., Serratia spp., Shigella spp., Stenotrophomonas maltophilia, Staphyloccocus aureus, Staphyloccocus epidermidis, Streptococcus pneumoniae, Streptococcus pyogenes, Streptococcus agalactiae, Yersina pestis, and Yersina enterocolitica, or the like; or Peptostreptococci asaccharolyticus, Peptostreptococci magnus, Peptostreptococci micros, Peptostreptococci prevotii, Porphyromonas asaccharolytica, Porphyromonas canoris, Porphyromonas gingivalis, Porphyromonas macaccae, Actinomyces israelii, Actinoinyces odontolyticus, Clostridium innocuum, Clostridium clostridioforme, Clostridium difficile, Bacteroides tectum, Bacteroides ureolyticus, Bacteroides gracilis (Campylobacter gracilis), Prevotella intermedia, Prevotella heparinolytica, Prevotella oris-buccae, Prevotella bivia, Prevotella melaminogenica, Fusobacterium naviforme, Fusobacterium necrophorum, Fusobacterium varium, Fusobacterium ulcerans, Fusobacterium russii, Bilophila wadsworthia, Haemophilus ducreyi; Calymmatobacterium granulomatis, or the like.

It is believed that the method can be particularly useful for treating a subject with an intracellular infection. It is generally believed in the art that NK cells are particularly effective against intracellular infections. Intracellular infections are those wherein a portion of the infecting pathogen resides within cells of the subject.

For example, intracellular infections can be caused by one or more bacteria selected from: Ehrlichia (e.g., obligate, intracellular bacteria that can appear as small cytoplasmic inclusions in lymphocytes and neutrophils such as Ehrlichia sennetsu, Ehrlichia canis, Ehrlichia chaffeensis, Ehrlichia phagocytophilia, or the like); Listeria (e.g., Listeria monocytogenes); Legionella (e.g., Legionella pneumophila); Rickettsiae (e.g., Rickettsiae prowazekii, Rickettsiae typhi (Rickettsiae mooseri), Rickettsiae rickettsii, Rickettsiae tsutsugamushi, Rickettsiae sibirica; Rickettsiae australis; Rickettsiae conorii; Rickettsiae akari; Rickettsiae burnetii); Chlamydia (e.g., Chlamydia psittaci; Chlamydia pneumoniae; Chlamydia trachomatis, or the like); Mycobacterium (Mycobacterium tuberculosis; Mycobacterium marinum; Mycobacterium Avium Complex; Mycobacterium bovis; Mycobacterium scrofulaceum; Mycobacterium ulcerans; Mycobacterium leprae (Leprosy, Hansen's Bacillus)); Brucella (e.g., Brucella melitensis; Brucella abortus; Brucella suis; Brucella canis); genus Coxiella (e.g., Coxiella burnetii); or the like. Thus, in some embodiments, the subject can have an intracellular bacterial infection caused by a bacterium selected from the genera Ehrlichia; Listeria; Legionella; Rickettsiae; Chlamydia; Mycobacterium; Brucella; and Coxiella.

In various embodiments, the subject can be treated for a bacterial infection from one or more upper respiratory tract bacteria. Examples of upper respiratory tract bacteria include those belonging genera such as Legionella, Pseudomonas, and the like. In some embodiments, the bacteria can be Pseudomonas aeruginosa. In particular embodiments, the bacteria can be Legionella pneumophila (e.g., including serogroups 1, 2, 3, 4, 5, 6, 7, 8, and the like), Legionella dumoffli, Legionella longbeacheae, Legionella micdadei, Legionella oakridgensis, Legionella feelei, Legionella anisa, Legionella sainthelensi, Legionella bozemanii, Legionella gormanii, Legionella wadsworthii, Legionella jordanis, or Legionella gormanii.

In some embodiments, the subject can be treated for a bacterial infection from one that causes acute bacterial exacerbation of chronic bronchitis (ABECB) in the subject. Typically, ABECB can be caused by Streptococcus pneumoniae, Haemophilus influenzae, Haemophilus parainfluenzae, or Moraxella catarrhalis.

In some embodiments, the subject can be treated for a bacterial infection from one that causes acute community acquired pneumonia (CAP) in the subject. Typically, CAP can be caused by Streptococcus pneumoniae, Haemophilus influenzae, Moraxella catarrhalis, Mycoplasma pneumoniae, Chlamydia pneumoniae, or Klebsiella pneumoniae. In a particular embodiment, the CAP can be caused by drug resistant bacteria, e.g., a multi-drug resistant strain of Streptococcus pneumoniae.

In various embodiments, the subject can be treated for a bacterial infection from Streptococcus pneumoniae, Haemophilus influenzae, Haemophilus parainfluenzae, Moraxella catarrhalis, Mycoplasma pneumoniae, Chlamydia pneumoniae, Klebsiella pneumoniae, Staphylococcus aureus, Streptococcus pyogenes, Acinetobacter lwoffi, Klebsiella oxytoca, Legionella pneumophila, or Proteus vulgaris.

In various embodiments, the subject can be treated for a bacterial infection from maxillary sinus pathogenic bacteria. As used herein, maxillary sinus pathogenic bacteria is a bacterial strain isolated from acute or chronic maxillary sinusitis, or, for example, a maxillary sinus isolate of Staphylococcus aureus, Streptococcus pneumoniae, Haemophilus spp., Moraxella catarrhalis, an anaerobic strain of non-fermentative Gram negative bacilli, Neisseria meningitides or β-haemolytic Streptococcus. In various embodiments, maxillary sinus pathogenic bacteria can include a bacterial strain isolated from acute or chronic maxillary sinusitis; a maxillary sinus isolate of Staphylococcus aureus, Streptococcus pneumoniae, Haemophilus spp., Moraxella catarrhalis, an anaerobic strain of non-fermentative Gram negative bacilli, Neisseria meningitidis, β-haemolytic Streptococcus, Haemophilus influenzae, an Enterobacteriaceae, a non-fermentative Gram negative bacilli, Streptococcus pneumoniae, Streptococcus pyogenes, a methicillin-resistant Staphylococcus spp., Legionella pneumophila, Mycoplasma spp. and Chlamydia spp., Haemophilus influenzae, Haemophilus parainfluenzae, Peptostreptococcus, Bacteroides spp., and Bacteroides urealyticus.

In various embodiments, the subject can be treated for a bacterial infection that causes a urinary tract infection (UTI) in the subject. Examples of UTIs include urethritis, cystitis, prostatitis, pyelonephritis (acute, chronic, and xanthogranulomatous), and hematogenous UTI (e.g., from bacteremia with virulent bacilli such as Salmonella, Staphylococcus aureus, and the like). Typically, UTIs can be caused by gram-negative aerobic bacteria, e.g., Escherichia (e.g., Escherichia coli), Klebsiella, Proteus, Enterobacter, Pseudomonas, and Serratia; gram-negative anaerobic bacteria; gram-positive bacteria, e.g., Enterococci (e.g., Enterococcus faecalis) and Staphylococcus sp (e.g., Staphylococcus saprophyticus, Staphylococcus aureus, and the like); Mycobacterium tuberculosis; and sexually transmitted bacterial infections (e.g., Chlamydia trachomatis, Neisseria gonorrhoeae, and the like).

In certain embodiments, it is believed the methods can be effective in treating infections from microorganisms that cause sexually transmitted diseases, for example, Treponema pallidum; Trichomonas vaginalis; Candidia (Candida albicans); Neisseria gonorrhoeae; Chlamydia trachomatis; Mycoplasma genitalium, Ureaplasma urealyticum; Haemophilus ducreyi; Calymmatobacterium granulomatis (formerly Donovania granulomatis); herpes simplex viruses (HSV-1 or HSV-2); human papillomavirus [HPV]; human immunodeficiency virus (HIV); various bacterial (Shigella, Campylobacter, or Salmonella), viral (hepatitis A), or parasitic (Giardia or amoeba, e.g., Entamoeba dispar (previously Entamoeba histolytica); or the like.

Thus, in various embodiments, the subject can have an infection resulting in upper respiratory tract bacterial infection, acute bacterial exacerbation of chronic bronchitis; acute community acquired pneumonia, maxillary sinus pathogenic bacteria; a urinary tract infection; or a sexually transmitted infection.

It is believed that the methods can be particularly effective for treating a subject with a viral infection. Thus, in various embodiments, a subject can be treated for infection from viruses such as Picornaviruses (e.g., Polio Virus, rhinoviruses and certain echoviruses and coxsackieviruses); Parvoviridae (Human Parvovirus B19); Hepatitis, e.g, Hepadnavirus (Hepatitis B); Papovavirus (JC Virus); Adenovirus (Human Adenovirus); Herpesvirus (e.g., Cytomegalovirus, Epstein Barr Virus (Mononucleosis), Mononucleosis-Like Syndrome, Roseola Infantum, Varicella Zoster Virus (Chicken Pox), Herpes Zoster (Shingles), Herpes Simplex Virus (Oral Herpes, Genital Herpes)), Poxvirus (Smallpox); Calicivirus (Norwalk Virus), Arbovirus (e.g., Togavirus (Rubella virus, Dengue virus), Flavivirus (Yellow Fever virus), Bunyavirus (California Encephalitis Virus), Reovirus (Rotavirus)); Coronavirus (Coronavirus); Retrovirus (Human Immunodeficiency Virus 1, Human Immunodeficiency Virus 2); Rhabdovirus (Rabies Virus), Filovirus (Marburg Virus, Ebola virus, other hemorrhagic viral diseases); Paramyxovirus (Measles Virus, Mumps Virus); Orthomyxovirus (Influenza Virus); Arenavirus (Lassa Fever); human T-cell Lymphotrophic virus type I and II (HTLV-I, HTLV II); human papillomavirus [HPV]; or the like. Thus, in various embodiments, the subject can have an infection caused by a virus selected from Picornavirus; Parvoviridae; Hepatitis virus; Papovavirus; Adenovirus; Herpesvirus, Poxvirus; Calicivirus; Arbovirus; Coronavirus; a Retrovirus; Rhabdovirus; Paramyxovirus; Orthomyxovirus; Arenavirus; human T-cell Lymphotrophic virus; human papillomavirus; and human immunodeficiency virus.

In some embodiments, a subject can be treated for infection from viruses or infections thereof such as human immunodeficiency virus-1, human immunodeficiency virus-2, Cytomegalovirus, Epstein Barr Virus, Mononucleosis-Like Syndrome, Roseola Infantum, Varicella Zoster Virus, Herpes Zoster, Herpes Simplex Virus, or hepatitis.

It is believed that the methods can be particularly effective for treating a subject with a parasitic infection. Thus, in various embodiments, a subject can be treated for infection from Plasmodia (e.g., Plasmodia falcipaium, Plasmodia vivax, Plasmodia ovale, and Plasmodia malariae, typically transmitted by anopheline mosquitoes); Leishmania (transmitted by sandflies and caused by obligate intracellular protozoa, e.g., Leishmania donovani, Leishmania infantum, Leishmania chagasi, Leishmania mexicana, Leishmania amazonensis, Leishmania venezuelensis, Leishmania tropica; Leishmania major; Leishmania aethiopica; and the subgenus Viannia, Leishmania Viannia braziliensis, Leishmania Viannia guyanensis, Leishmania Viannia panamensis, and Leishmania Viannia peruviana); Trypanosoma (e.g., sleeping sickness caused by Trypanosoma bricei gambiense, and Trypanosoma brucei rhodesiense); amoebas of the genera Naegleria or Acanthamoeba; pathogens such as genus Entamoeba (Entamoeba histolytica and Entamoeba dispar); Giardia lamblia; Cryptosporidium; Isospora; Cyclospora; Microsporidia; Ascaris lumbricoides; infection with blood flukes of the genus Schistosoma (e.g.; S. haematobium; S. mansoni; S. japonicum; S. mekongi; S. intercalatum); Toxoplasmosis (e.g., Toxoplasma gondii); Treponema pallidum; Trichomonas vaginalis; or the like.

In some embodiments, the subject can have an infection caused by a protozoa selected from Toxoplasma gondii, Trypanosoma brucei gambiense, Trypanosoma brucei rhodesiense, Leishmania donovani, Leishmania infantum, Leishmania chagasi, Leishmania mexicana, Leishmania amazonensis, Leishmania venezuelensis, Leishmania tropica; Leishmania major; Leishmania aethiopica; and the subgenus Viannia, Leishmania Viannia braziliensis, Leishmania Viannia guyanensis, Leishmania Viannia panamensis, Leishmania Viannia peruviana, Plasmodia falciparum, Plasmodia vivax, Plasmodia ovale, and Plasmodia malariae.

In the last century, antibiotics were developed that led to significant reductions in mortality. Unfortunately, widespread use has led to the rise of antibiotic resistant bacteria, e.g., methicillin resistant Staphyloccocus aureus (MRSA), vancomycin resistant enterococci (VRE), and penicillin-resistant Streptococcus pneumoniae (PRSP). Some bacteria are resistant to a range of antibiotics, e.g., strains of Mycobacterium tuberculosis resist isoniazid, rifampin, ethambutol, streptomycin, ethionamide, kanamycin, and rifabutin. In addition to resistance, global travel has spread relatively unknown bacteria from isolated areas to new populations. Furthermore, there is the threat of bacteria as biological weapons. These bacteria may not be easily treated with existing antibiotics.

It is believed that the methods can be particularly effective for treating a subject for drug-resistant pathogens, for example, drug resistant bacteria, or pathogens for which no drugs are available, e.g., many viruses. Without wishing to be bound by theory, it is believed that because the methods can act by increasing NK cell activity, and thus the NK cells can kill infective microorganisms or infected cells separately from any direct action of the compounds on the pathogen or infected cells. Thus, it is believed that the methods can have at least one mode of action that is separate from typical anti-infective drugs such as antibiotics which can typically act directly on the bacteria themselves.

Drug resistant pathogens can be resistant to at least one and typically multiple agents, for example, drug resistant bacteria can be resistant to one antibiotic, or typically at least two antibiotics such as penicillin, Methicillin, second generation cephalosporins (e.g., cefuroxime, and the like), macrolides, tetracyclines, trimethoprim/methoxazole, vancomycin, or the like. For example, in some embodiments, a subject can be treated for bacteria selected from a strain of multiple drug resistant Streptococcus pneumoniae (MDRSP, previously known as penicillin resistant Streptococcus pneumoniae, PRSP), vancomycin resistant Enterococcus, methicillin resistant Staphylococcus Aureus, penicillin resistant Pneumococcus, antibiotic resistant Salmonella, resistant and multi-resistant Neisseria Gonorrhea (e.g., resistant to one, two or more of tetracycline, penicillin, fluoroquinolones, cephalosporins, ceftriaxone (Rocephin), Cefixime (Suprax), Azithromycin, or the like), and resistant and multi-resistant Tuberculosis (e.g., resistant to one, two or more of Isoniazid, Rifampin, Ethambutol, Pyrazinamide, Aminoglycoside, Capreomycin, Ciprofloxacin, Ofloxacin, gemifloxacin, Cycloserine, Ethionamide, para-aminosalicylic acid or the like).

In some embodiments, NK activity can be increased in subjects that have an immunodeficiency. In various embodiments, this can be due to decreased or deficient NK cell activity. In some embodiments, the immunodeficiency can be any known immunodeficiency, even those that do not directly impact NK cells. Without wishing to be bound by theory, it is believed that boosting NK cell activity can augment immune function in many immunodeficiency conditions to “make-up” at least in part, for aspects of immunodeficiency separate from those aspects directly concerned with NK cell activity.

In various embodiments, immunodeficiency disorders can include disorders with increased susceptibility to infection, for example, one or more disorders selected from: circulatory and systemic disorders (sickle cell disease, diabetes mellitus, nephrosis, varicose veins, congenital cardiac defects); obstructive disorders (ureteral or urethral stenosis, bronchial asthma, bronchiectasis, allergic rhinitis, blocked Eustachian tubes); integumentary defects (eczema, burns, skull fractures, midline sinus tracts, ciliary abnormalities); primary immunodeficiencies (X-linked agammaglobulinemia, DiGeorge anomaly, chronic granulomatous disease, C3 deficiency); secondary immunodeficiencies (malnutrition, prematurity, lymphoma, splenectomy, uremia, immunosuppressive therapy, protein-losing enteropathy, chronic viral diseases); unusual microbiologic factors (antibiotic overgrowth, chronic infections with resistant organism, continuous reinfection (contaminated water supply, infectious contact, contaminated inhalation therapy equipment)); foreign bodies, trauma (ventricular shunts, central venous catheter, artificial heart valves, urinary catheter, aspirated foreign bodies) allogeneic transplant, graft-versus-host disease, uterine dysfunction (e.g., endometriosis), or the like.

In various embodiments, immunodeficiency disorders can include for example, transient hypogammaglobulinemia of infancy, selective IgA deficiency, X-linked agammaglobulinemian (Bruton's Agammaglobulinemia; Congenital Agammaglobulinemia), common variable immunodeficiency (Acquired Agammaglobulinemia), hyper-IgM immunodeficiency, IgG subclass deficiency, chronic mucocutaneous Candidiasis, combined immunodeficiency, Wiskott-Aldrich syndrome, ataxia-telangiectasia, X-linked lymphoproliferative syndrome, hyper-IgE syndrome (Job-Buckley Syndrome), chronic granulotomatous disease, leukocyte adhesion deficiency (MAC-1/LFA-1/CR3 deficiency), or the like

In various embodiments, immunodeficiency disorders can include primary immunodeficiency disorders for example: B-cell (antibody) deficiencies (X-linked agammaglobulinemia; Ig deficiency with hyper-IgM (XL); IgA deficiency); IgG subclass deficiencies, Antibody deficiency with normal or elevated Igs, Immunodeficiency with theymoma, Common variable immunodeficiency, Transient hypogammaglobulinemia of infancy); T-cell (cellular) deficiencies (Predominant T-cell deficiency: DiGeorge anomaly, Chronic mucocutaneous candidiasis, Combined immunodeficiency with Igs (Nezelof syndrome), Nucleoside phosphorylase deficiency (AR), Natural killer cell deficiency, Idiopathic CD4 lymphocytopenia, Combined T- and B-cell deficiencies: Severe combined immunodeficiency (AR or XL), Adenosine deaminase deficiency (AR), Reticular dysgenesis, Bare lymphocyte syndrome, Ataxia-telangiectasia (AR), Wiskott-Aldrich syndrome (XL), Short-limbed dwarfism, XL lymphoproliferative syndrome); Phagocytic disorders (Defects of cell movement: Hyperimmunoglobulinemia E syndrome, Leukocyte adhesion defect type 1 (AR), Defects of microbicidal activity: Chronic granulomatous disease (XL or AR), Neutrophil G6PD deficiency, Myeloperoxidase deficiency (AR), Chediak-Higashi syndrome (AR)); Complement disorders (Defects of complement components: C1q deficiency, Defects of control proteins: Cl inhibitor deficiency (D1), Factor I (C3b inactivator) deficiency (ACD), Factor H deficiency (ACD), Factor D deficiency (ACD), Properdin deficiency (XL)); or the like

In various embodiments, immunodeficiency disorders can include secondary immunodeficiency disorders, for example, one or more conditions selected from: Premature and newborn infants (Physiologic immunodeficiency due to immaturity of immune system); Hereditary and metabolic diseases (Chromosome abnormalities (e.g., Down syndrome), Uremia, Diabetes (i.e., complications from diabetes such as gangrene associated with peripheral circulatory and nerve dysfunction), Malnutrition, Vitamin and mineral deficiencies, Protein-losing enteropathies, Nephrotic syndrome, Myotonic dystrophy, Sickle cell disease); Immunosuppressive agents (Radiation, Immunosuppressive drugs, Corticosteroids, Anti-lymphocyte or anti-thymocyte globulin, Anti-T-cell monoclonal antibodies); Infectious diseases (Congenital rubella, Viral exanthems (e.g., measles, varicella), HIV infection, Cytomegalovirus infection, Infectious mononucleosis, Acute bacterial disease, Severe mycobacterial or fungal disease); Infiltrative and hematologic diseases (Histiocytosis, Sarcoidosis, Hodgkin's disease and lymphoma, Leukemia, Myeloma, Agranulocytosis and aplastic anemia); Surgery and trauma (Burns, Splenectomy, Anesthesia, wounds); and Miscellaneous (SLE, Chronic active hepatitis, Alcoholic cirrhosis, Aging, Anticonvulsive drugs, Graft-vs.-host disease); or the like.

In certain embodiments, the subject can be treated for burns or wounds. Typically, such a wound or burn is a severe injury that places a significant burden on the subject's immune defenses. For example, in some embodiments, the subject is treated for a second or third degree burn covering at least about 5%, 10%, 15%, 20%, 25%, 30%, 40%, 50%, 75%, or more of the surface area of the subject's body. Also, in some embodiments, the subject is treated for a wound or wounds, e.g., an open wound of at least about 1 cm², 2 cm², 5 cm², 10 cm², 20 cm², 50 cm² or larger, or 1%, 2%, 3%, 4%, 5%, 10%, 15%, or more of the surface area of the subject's body; or one or more incisions penetrating the skin totaling at least 1 cm, 2 cm, 3 cm, 4 cm, 5 cm, 7 cm, 10 cm, 20 cm, 25 cm, 50 cm in length; an amputation; and the like.

In various embodiments, the subject can have an infection caused by antibiotic resistant bacteria. In some embodiments, the subject can have an infection caused by a bacterium selected from multiple drug resistant Streptococcus pneumoniae, vancomycin resistant Enterococcus, methicillin resistant Staphylococcus Aureus, penicillin resistant Pneumococcus, antibiotic resistant Salmonella, resistant/multi-resistant Neisseria Gonorrhea, and resistant/multi-resistant Tuberculosis. In some embodiments, the subject can have a bacterial infection resistant to at least one antibiotic selected from penicillin, Methicillin, second generation cephalosporins, macrolides, tetracyclines, trimethoprim/methoxazole, vancomycin, tetracycline, fluoroquinolones, ceftriaxone, Cefixime, Azithromycin, Isoniazid, Rifampin, Ethambutol, Pyrazinamide, Aminoglycoside, Capreomycin, Ciprofloxacin, Ofloxacin, gemifloxacin, Cycloserine, Ethionamide, and para-aminosalicylic acid.

Thus, various embodiments, the subject can have an immunodeficiency disorder. In some embodiments, the subject can have a primary immunodeficiency disorder. In some embodiments, the subject can have a secondary immunodeficiency disorder.

In some embodiments, immunodeficiency disorders can include uremia, diabetes (infective complications thereof, malnutrition, vitamin and mineral deficiencies, protein-losing enteropathies, nephrotic syndrome, myotonic dystrophy, sickle cell disease; or the like.

In some embodiments, immunodeficiency disorders can include immunosuppressive agents, e.g., radiation, immunosuppressive drugs, corticosteroids, anti-lymphocyte or anti-thymocyte globulin, anti-T-cell monoclonal antibodies; or the like.

In some embodiments, immunodeficiency disorders can include surgery and trauma, e.g., burns, splenectomy, anesthesia, wounds, implanted medical devices; or the like.

In some embodiments, immunodeficiency disorders can include chronic fatigue syndrome (chronic fatigue immune dysfunction syndrome); Epstein-Barr virus infection, post viral fatigue syndrome, post-transplantation syndrome (host-graft disease), exposure to nitric oxide synthase inhibitors, aging, severe combined immunodeficiency, variable immunodeficiency syndrome, and the like.

As used herein, a “pharmaceutical composition” can be a formulation containing the disclosed compounds, in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition can be an effective amount and can be varied according to the particular treatment involved. It may be appreciated that it can be necessary to make routine variations to the dosage depending on the age and condition of the patient. The dosage can also depend on the route of administration. A variety of routes are contemplated, including topical, oral, pulmonary, rectal, vaginal, parenternal, including transdermal, subcutaneous, intravenous, intramuscular, intraperitoneal and intranasal.

The compounds described herein, and the pharmaceutically acceptable salts thereof can be used in pharmaceutical preparations in combination with a pharmaceutically acceptable carrier or diluent. Suitable pharmaceutically acceptable carriers include inert solid fillers or diluents and sterile aqueous or organic solutions. The compounds can be present in such pharmaceutical compositions in amounts sufficient to provide the desired dosage amount in the range described herein. Techniques for formulation and administration of the disclosed compounds of the invention can be found in Remington: the Science and Practice of Pharmacy, 19^{th} edition, Mack Publishing Co., Easton, Pa. (1995).

For oral administration, the disclosed compounds or salts thereof can be combined with a suitable solid or liquid carrier or diluent to form capsules, tablets, pills, powders, syrups, solutions, suspensions, or the like.

The tablets, pills, capsules, and the like can contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials can be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration, the bis(thio-hydrazide) amides can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, the compounds may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Typically, they can be implanted in, or applied to, the microenvironment of an affected organ or tissue, for example, a membrane impregnated with the disclosed compound can be applied to an open wound or burn injury. Thus, for example, the compounds may be formulated with suitable polymeric or hydrophobic materials, for example, as an emulsion in an acceptable oil, or ion exchange resins, or as sparingly soluble derivatives, for example, as a sparingly soluble salt.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, a cream formulation can be administer to a wound site, or a bandage may be impregnated with a formulation, and the like.

For rectal administration, suitable pharmaceutical compositions are, for example, topical preparations, suppositories or enemas.

For vaginal administration, suitable pharmaceutical compositions are, for example, topical preparations, pessaries, tampons, creams, gels, pastes, foams or sprays.

In addition, the compounds may also be formulated to deliver the active agent by pulmonary administration, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

A drug delivery device for delivering aerosols can comprise a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. Where the liquid carrier is used, the formulation may be administered as a nasal spray or drops and may include oil or aqueous solutions of the active ingredients.

In addition to the formulations described above, a formulation can optionally include, or be co-administered with one or more additional drugs, e.g., other antifungals, anti-inflammatories, anti-biotics, antivirals, immunomodulators, antiprotozoals, steroids, decongestants, bronchodialators, antihistamines, anticancer agents, and the like. For example, the disclosed compound can be co-administered with drugs such as such as ibuprofen, prednisone (corticosteroid) pentoxifylline, Amphotericin B, Fluconazole, Ketoconazol, Itraconazol, penicillin, ampicillin, amoxicillin, and the like. The formulation may also contain preserving agents, solubilizing agents, chemical buffers, surfactants, emulsifiers, colorants, odorants and sweeteners.

Hsp70-responsive disorders excluded by proviso from various embodiments include any such disorder identified in Barsoum, U.S. Provisional Application No. 60/629,595 (Attorney's Docket No. 3211.1017-000); filed Nov. 19, 2004, the entire teachings of which are incorporated by reference. As used herein, a non-infective heat shock protein 70 (Hsp70) responsive disorder, e.g., the Hsp70 disorders excluded by proviso from various embodiments, can be a medical condition wherein stressed cells can be treated by increased Hsp70 expression. Such disorders can be caused by a wide variety of cellular stressors, including, but not limited to Alzheimers' disease; Huntington's disease; Parkinson's disease; spinal/bulbar muscular atrophy (e.g., Kennedy's disease), spinocerebellar ataxic disorders, and other neuromuscular atrophies; familial amyotrophic lateral sclerosis; ischemia; seizure; hypothermia; hyperthermia; burn trauma; atherosclerosis; radiation exposure; glaucoma; toxin exposure; mechanical injury; inflammation; and the like.

As used herein, “Hsp70” includes each member of the family of heat shock proteins having a mass of about 70-kiloDaltons, including forms such as constituitive, cognate, cell-specific, glucose-regulated, inducible, etc. Examples of specific Hsp70 proteins include hsp70, hsp70hom; hsc70; Grp78/BiP; mt-hsp70/Grp75, and the like). Typically, the disclosed methods increase expression of inducible Hsp70. Functionally, the 70-kDa HSP (HSP70) family is a group of chaperones that assist in the folding, transport, and assembly of proteins in the cytoplasm, mitochondria, and endoplasmic reticulum. In humans, the Hsp70 family encompasses at least 11 genes encoding a group of highly related proteins. See, for example, Tavaria, et al., Cell Stress Chaperones, 1996; 1(1):23-28; Todryk, et al., Immunology. 2003, 110(1): 1-9; and Georgopoulos and Welch, Annu Rev Cell Biol. 1993; 9:601-634; the entire teachings of these documents are incorporated herein by reference.

An example of Hsp70 disorders excluded by proviso from various embodiments can include a neurodegenerative disorder. As used herein, a neurodegenerative disorder involves degradation of neurons such as cereberal, spinal, and peripheral neurons (e.g., at neuromuscular junctions), more typically degradation of cerebral and spinal neurons. Neurodegenerative disorders can include Alzheimers' disease; Huntington's disease; Parkinson's disease; spinal/bulbar muscular atrophy and other neuromuscular atrophies; and familial amyotrophic lateral sclerosis or other diseases associated with superoxide dismutase (SOD) mutations. Neurodegenerative disorders can also include degradation of neurons caused by ischemia, seizure, thermal stress, radiation, toxin exposure, infection, injury, and the like.

Other examples of Hsp70 disorders excluded by proviso from various embodiments can include a disorder of protein aggregation/misfolding, such as Alzheimers' disease; Huntington's disease; Parkinson's disease; and the like.

Additional examples of Hsp70 disorders excluded by proviso from various embodiments can include ischemia. Ischemia can damage tissue through multiple routes, including oxygen depletion, glucose depletion, oxidative stress upon reperfusion, and/or glutamate toxicity, and the like. Ischemia can result from an endogenous condition (e.g., stroke, heart attack, and the like), from accidental mechanical injury, from surgical injury (e.g., reperfusion stress on transplanted organs), and the like. Alternatively, tissues that can be damaged by ischemia include neurons, cardiac muscle, liver tissue, skeletal muscle, kidney tissue, pulmonary tissue, pancreatic tissue, and the like.

Also, examples of Hsp70 disorders excluded by proviso from various embodiments can include seizure, e.g., eplileptic seizure, injury-induced seizure, chemically-induced seizure, and the like.

More examples of Hsp70 disorders excluded by proviso from various embodiments can include disorders due to thermal stress. Thermal stress includes hyperthermia (e.g., from fever, heat stroke, burns, and the like) and hypothermia.

Further examples of Hsp70 disorders excluded by proviso from various embodiments can include radiation damage, e.g., due to visible light, ultraviolet light, microwaves, cosmic rays, alpha radiation, beta radiation, gamma radiation, X-rays, and the like. For example, the damage could be radiation damage to non-cancerous tissue in a subject treated for cancer by radiation therapy.

Certain examples of Hsp70 disorders excluded by proviso from various embodiments can include mechanical injury, e.g., trauma from surgery, accidents, certain disease conditions (e.g., pressure damage in glaucoma) and the like.

Particular examples of Hsp70 disorders excluded by proviso from various embodiments can include exposure to a toxin. e.g., exposure to a neurotoxin selected from methamphetamine; antiretroviral HIV therapeutics (e.g., nucleoside reverse transcriptase inhibitors; heavy metals (e.g., mercury, lead, arsenic, cadmium, compounds thereof, and the like), amino acid analogs, chemical oxidants, ethanol, glutamate, metabolic inhibitors, antibiotics, and the like.

Cancer is excluded from the present invention. Examples include those identified in Koya, et al, U.S. Pat. Nos. 6,800,660, issued October 5; 6,762,204, issued, Jul. 13, 2004; and Koya, et al U.S. application Ser. No. 10/758,589; Filed: Jan. 15, 2004; the entire teachings of which are incorporated by reference. For example, such cancers can be human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

Other examples of cancer excluded by proviso from various embodiments include leukemias include acute and/or chronic leukemias, e.g., lymphocytic leukemia (e.g., as exemplified by the p388 (murine) cell line), large granular lymphocytic leukemia, and lymphoblastic leukemia; T-cell leukemias, e.g., T-cell leukemia (e.g., as exemplified by the CEM, Jurkat, and HSB-2 (acute), YAC-1(murine) cell lines), T-lymphocytic leukemia, and T-lymphoblastic leukemia; B cell leukemia (e.g., as exemplified by the SB (acute) cell line), and B-lymphocytic leukemia; mixed cell leukemias, e.g., B and T cell leukemia and B and T lymphocytic leukemia; myeloid leukemias, e.g., granulocytic leukemia, myelocytic leukemia (e.g., as exemplified by the HL-60 (promyelocyte) cell line), and myelogenous leukemia (e.g., as exemplified by the K562(chronic)cell line); neutrophilic leukemia; eosinophilic leukemia; monocytic leukemia (e.g., as exemplified by the THP-1 (acute) cell line); myelomonocytic leukemia; Naegeli-type myeloid leukemia; and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B. C. Decker Inc. (2000). The entire teachings of the preceding references are incorporated herein by reference.

Other examples of cancer excluded by proviso from various embodiments include non-solid tumors such as multiple myeloma, T-leukemia (e.g., as exemplified by Jurkat and CEM cell lines); B-leukemia (e.g., as exemplified by the SB cell line); promyelocytes (e.g., as exemplified by the HL-60 cell line); uterine sarcoma (e.g., as exemplified by the MES-SA cell line); monocytic leukemia (e.g., as exemplified by the THP-4(acute) cell line); and lymphoma (e.g., as exemplified by the U937 cell line).

Other examples of cancer excluded by proviso from various embodiments include colon cancer, pancreatic cancer, melanoma, renal cancer, sarcoma, breast cancer, ovarian cancer, lung cancer. stomach cancer, bladder cancer and cervical cancer.

Other examples of cancer excluded by proviso from various embodiments include cancer has become “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. Drug resistant tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

Proliferative cell disorders are excluded from the present invention. Examples include those disorders identified in Sherman et al, U.S. Provisional Application Ser. No. 60/610,270; filed Sep. 16, 2004 (Attorney's docket No. 3211.1015-000), the entire teachings of which are incorporated by reference. For example, non-cancerous proliferative disorders excluded by proviso from various embodiments include smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis, desmoid tumors and the like. Non-cancerous proliferative disorders excluded by proviso from various embodiments also include smooth muscle cell proliferation, e.g., proliferative vascular disorders, for example, intimal smooth muscle cell hyperplasia, restenosis and vascular occlusion, particularly stenosis following biologically- or mechanically-mediated vascular injury, e.g., vascular injury associated with balloon angioplasty or vascular stenosis. Moreover, intimal smooth muscle cell hyperplasia can include hyperplasia in smooth muscle other than the vasculature, e.g., hyperplasia in bile duct blockage, in bronchial airways of the lung in asthma patients, in the kidneys of patients with renal interstitial fibrosis, and the like. Non-cancerous proliferative disorders excluded by proviso from various embodiments also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

Proteasome inhibitor responsive disorders excluded from the present invention. Examples include those disorders identified in Mei Zhang, et al, U.S. Provisional Application Ser. No. 60/629,858; filed: Nov. 19, 2004, (Attorney's Docket No. 3211.1018-000), the entire teachings of which are incorporated by reference. Such conditions include for example, the above cancer and non-cancerous proliferative conditions, conditions marked by excessive or accelerated protein degradation, and Hsp70-responsive disorders. Additional examples of proteasome inhibitor responsive disorders excluded by proviso from various embodiments include muscle-wasting diseases (e.g., fever, muscle disuse (atrophy) and denervation, nerve injury, fasting, renal failure associated with acidosis, hepatic failure, uremia, diabetes, and sepsis), skeletal system disorders resulting from bone loss or low bone density (e.g., closed fractures, open fractures, non-union fractures, age-related osteoporosis, post-menopausal osteoporosis, glucocorticoid-induced osteoporosis, disuse osteoporosis, arthritis), growth deficiencies (e.g., periodontal disease and defects, cartilage defects or disorders), disorders of hair growth (e.g., male pattern baldness, alopecia caused by chemotherapy, hair thinning resulting from aging, genetic disorders resulting in deficiency of hair coverage), dry-eye disorders (e.g., excessive inflammation in relevant ocular tissues, such as the lacrimal and meibomian glands, dry eye associated with refractive surgery (e.g., LASIK surgery)) and cystic fibrosis.

### EXEMPLIFICATION

### Example 1

### Measurement of Heat Shock Protein 70 (Hsp70)

Plasma Hsp70 was measured by a sandwich ELISA kit (Stressgen Bioreagents Victoria, British Columbia, CANADA) according to a modified protocol in house. In brief, Hsp70 in plasma specimens and serial concentrations of Hsp70 standard were captured onto 96-well plate on which anti-Hsp70 antibody was coated. Then captured Hsp70 was detected with a biotinylated anti-Hsp70 antibody followed by incubation with europium-conjugated streptavidin. After each incubation unbound materials were removed by washing. Finally, antibody-Hsp70 complex was measured by time resolved fluorometry of europium. Concentration of Hsp70 was calculated from a standard curve.

### Example 2

### Measurement of Natural Killer Cell Cytotoxic Activity

The following procedure can be employed to assay NK cell activity in a subject. The procedure is adapted from Kantakamalakul W, Jaroenpool J, Pattanapanyasat K. A novel enhanced green fluorescent protein (EGFP)-K562 flow cytometric method for measuring natural killer (NK) cell cytotoxic activity. J Immunol Methods. 2003 Jan. 15; 272:189-197, the entire teachings of which are incorporated herein by reference.

Materials and methods: Human erythroleukaemic cell line, K562, was obtained from American Type Culture Collection (CCL-243, American Type Culture Collection, Manassas, Va.), and cultured in RPMI-1640 medium (Cat#1875-093Gibco Invitrogen Corp, Carlsbad, Calif.) supplemented with 10% heat inactivated fetal calf serum (Gibco), 2 mM L-glutamin, 100 μg/ml streptomycin and 100 IU/ml penicillin at 37° C. with 5% CO₂. K562 cells were transduced with retroviral vector which encode green fluorescent protein (eGFP). Stable cell line was selected with antibiotic, G418. About 99.6% G418 resistant cells were eGFP positive after section.

The subject's peripheral blood mononuclear cells (PBMCs) were prepared by clinical study sites and received in BD Vacutainer Cell Preparation Tube with sodium heparin (Product Number: 362753, Becton Dickinson, Franklin Lakes, N.J.).

Two-fold serial dilution of 800 μl effector cells (patient's PBMC) starting at concentration of 1×10⁶ cells/mL were put into four individual polystyrene 12×75-mm tubes. Log phase growing target cells (K562/eGFP) were adjusted with growth medium (RPMI-1640) to a concentration of 1×10⁵ cells/mL and 100 μL targets then added into the tubes to provide effector/target (E/T) ratios of 80:1, 40:1, 20:1, 10:1. Effector cells alone and target cells alone were used as controls. All tubes were incubated at 37° C. with 5% CO₂ for about 3.5 hr. Ten microliters of propidium iodide (PI) at a concentration of 1 mg/mL was added t each tube including effector and target control tubes and then incubated at room temperature for 15 min.

Cytotoxic activity was analyzed with a FACSCalibur flow cytometer (Becton Dickinson). Linear amplification of the forward and side scatter (FSC/SSC) signals, as well as logarithmic amplification of eGFP and PI emission in green and red fluorescence were obtained. Ten thousand events per sample tube with no gating for acquisition were collected for analysis. Data analysis for two-parameter dot plots for eGFP versus PI was performed using CELLQuest (Becton Dickinson Biosciences) software to enumerate live and dead target cells. Debris and dead cells were excluded by setting a threshold of forward light scatter.

### Example 3

### The Disclosed Combination Therapy Induces Hsp70

A Phase I trial was conducted for combined administration of a bis(thio-hydrazide) amide (Compound (1)) and a taxane (paclitaxel) to human subjects with various advanced solid tumors. Compound (1) and paclitaxel were co-administered intravenously over 3 hours every 3 weeks. Starting doses were 44 milligrams/meter² (mg/m2, or 110 micromoles/meter² (μmol/m2)) Compound (1) and 135 mg/m2 (158 μmol/m2) paclitaxel. Paclitaxel was then increased to 175 mg/m2 (205 μmol/m2), followed by escalation of Compound (1) to establish the maximum tolerated dose based on first cycle toxicity in 3 to 6 patients at each dose level. Pharmacokinetic (PK) studies were performed during cycle 1 using liquid chromatography/mass spectrometry (LC/MS) to measure both compounds in plasma. Heat shock protein 70 (Hsp70) was measured in plasma before and after treatment. 35 patients were evaluated at 8 dose levels, including paclitaxel at 135 mg/m2 (158 μmol/m2) and Compound (1) at 44 mg/m2, and paclitaxel at 175 mg/m2 (205 μmol/m2) and Compound (1) at a doses ranging among 44-525 mg/m2 (110-1311 μmol/m2). Table 1 shows the eight different doses #1-#8 in mg/m² and μmol/m².

TABLE 1

| | #1 | #2 | #3 | #4 | #5 | #6 | #7 | #8 |
|---|---|---|---|---|---|---|---|---|
| Compound (1), | 44 | 44 | 88 | 175 | 263 | 350 | 438 | 525 |
| mg/m² | | | | | | | | |
| Compound (1), | 110 | 110 | 220 | 437 | 657 | 874 | 1094 | 1311 |
| μmol/m² | | | | | | | | |
| Paclitaxel, | 135 | 175 | 175 | 175 | 175 | 175 | 175 | 175 |
| mg/m² | | | | | | | | |
| Paclitaxel, | 158 | 205 | 205 | 205 | 205 | 205 | 205 | 205 |
| μmol/m² | | | | | | | | |

No serious effects specifically attributable to Compound (1) were observed. Paclitaxel dose limiting toxicities occurred in a single patient in each of the top three dose levels (neutropenia, arthralgia, and febrile neutropenia with mucositis) resulting in cohort expansion. Compound (1) exhibited linear PK that was unaffected by paclitaxel dose, and was rapidly eliminated from plasma with terminal-phase half life of 0.94±0.23 hours (h) and total body clearance of 28±8 Liters/hour/meter² (L/h/m²). Its apparent volume of distribution was comparable to total body water (Vₛₛ23±16 L/m²). Paclitaxel PK appeared to be moderately dependent on the Compound (1) dose, as indicated by a significant trend toward decreasing clearance, and increase in peak plasma concentration and Vₛₛ, but without affecting the terminal phase half-life. These observations are consistent with competitive inhibition of paclitaxel hepatic metabolism. Increased toxicity at higher dose levels was consistent with a moderate increase in systemic exposure to paclitaxel. Induction of Hsp70 protein in plasma was dose dependent, peaking between about 8 hours to about 24 hours after dosing.

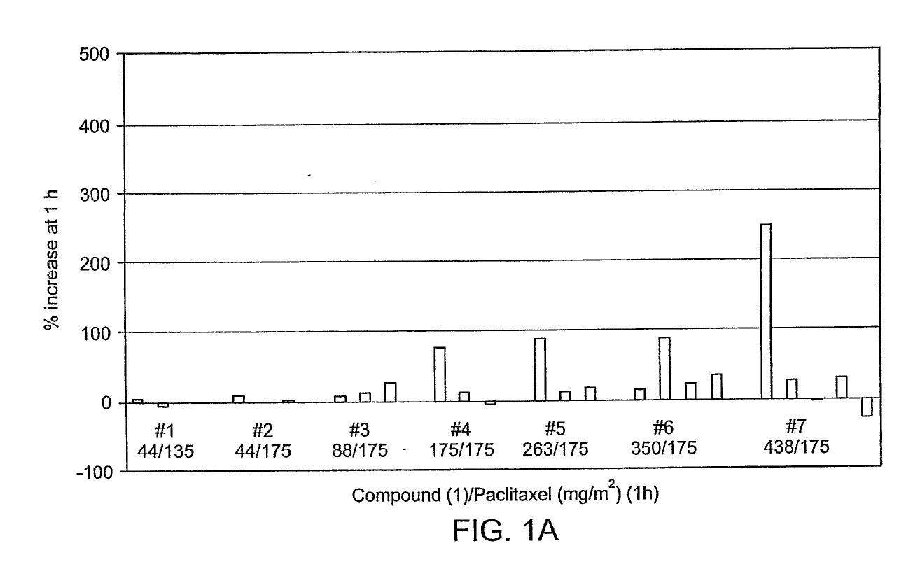

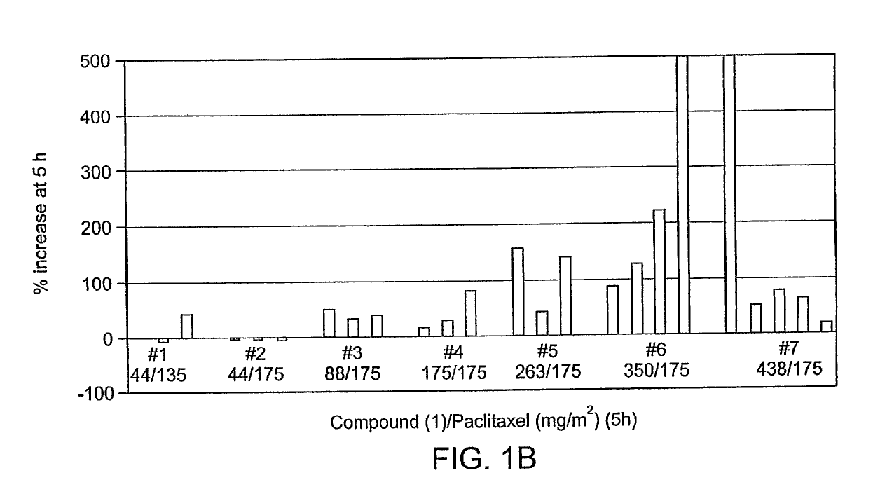

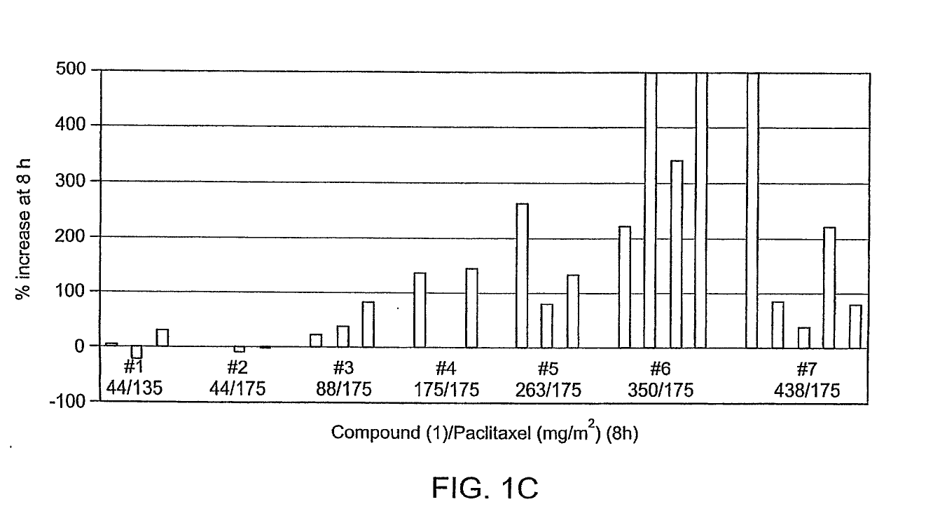

FIGS. 1A, 1B, and 1C are bar graphs showing the percent increase in Hsp70 plasma levels associated with administration of the Compound (1)/paclitaxel combination therapy at 1 hour (FIG. 1A), 5 hours (FIG. 1B), and 8 hours (FIG. 1C) after administration. Significant rises in Hsp70 levels occurred for at least one patient at the 88 mg/m2 (220 μmol/m2) Compound (1) dose, where Hsp70 levels nearly doubled in a percent increase of about 90%. At the 175 mg/m2 (437 μmol/m2) Compound (1) dose, Hsp70 concentrations more than doubled in two patients; at the 263 mg/m2 (657 μmol/m2) Compound (1) dose, Hsp70 concentrations roughly doubled in two patients and increased by more than 250% in a third patient; at the 350 mg/m2 (874 μmol/m2) Compound (1) dose, Hsp70 concentrations increased more than 200% in all patients and increased by as much as 500% in two patients; at the 438 mg/m2 (1094 μmol/m2) Compound (1) dose, Hsp70 concentrations roughly doubled in two patients, increased by over 2005 in one patient, and increased by as much as 500% in another patient.

Time to progression will be measured as the time from patient randomization to the time the patient is first recorded as having tumor progression according to the RECIST (Response Evaluation Criteria in Solid Tumors Group) criteria; see Therasse P, Arbuck S G, Eisenhauer E A, Wanders J, Kaplan R S, Rubinstein L, et al. New guidelines to evaluate the response to treatment in solid tumors. J Natl Cancer Inst 2000; 92:205-16, the entire teachings of which are incorporated by reference. Death from any cause will be considered as progressed.

Time to progression can be performed on the randomized sample as well as the efficacy sample. Treatment groups can be compared using the log-rank test and Kaplan-Meier curves of time to progression can be presented.

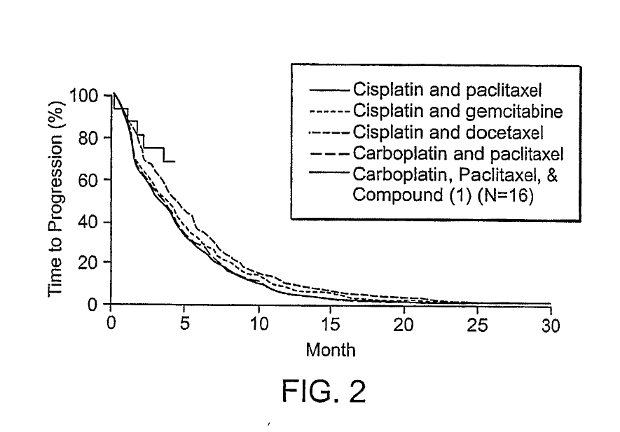

FIG. 2 is a Kaplan-Meier graph of time-to-progression (resumption of cancer growth) in studies of various combinations of platinum anticancer drugs and taxanes. Also shown is the disclosed combination of a bisthiohydrazide (Compound (1)), a taxane (paclitaxel) and also a platinum anticancer drug, carboplatin. The preliminary data in show that the disclosed method is superior to the platin/taxane combination alone.

Thus, the combination of a bi(thio-hydrazide) amide and taxane dramatically increased plasma Hsp70 levels in patients, giving significant increases for patients at a combined paclitaxel dose of 175 mg/m2 (205 μmol/m2) and Compound (1) doses ranging from 88 through 438 mg/m2 (220-1094 μmol/m2). Moreover, the combination was well-tolerated, with adverse events consistent with those expected for paclitaxel alone.

### Example 4

### A Phase 2 Study Shows the Disclosed Combination Therapy with Carboplatin is Effective for Treating Non-Small Cell Lung Carcinoma

The following study of Compound (1) and paclitaxel in patients with non-small cell lung carcinoma was initiated based on the biological activity shown by the results of the above Phase I study, where the combined administration Compound (1) and paclitaxel led to dose-related Hsp70 induction.

Phase 1 (safety/PK/MTD (maximum tolerated dose) was followed by a Phase 2 randomized two arm portion. Two dose levels were evaluated in Phase 1.

Cohort 1 was dosed with carboplatin AUC (area under the curve) 6, paclitaxel 175 mg/m2 and Compound (1) 233 mg/m2. If the maximum tolerated dose was not observed, Cohort 2 was enrolled with carboplatin AUC 6, paclitaxel 200 mg/m2 and Compound (1) 266 mg/m2.

Dosing was IV q 3 weeks for up to 6 cycles in the absence of dose-limiting toxicity or progression. In the phase 2 portion, 86 patients are planned to be randomized 1:1 to carboplatin AUC 6+paclitaxel 200 mg/m2 IV q 3 weeks or carboplatin AUC 6, paclitaxel 200 mg/m2 and Compound (1) 266 mg/m2. The phase 2 primary endpoint is time to progression, with secondary endpoints of response rate, survival, and quality of life. Study pharmacodynamic parameters include NK cell activity and Hsp70 level.

Sixteen patients were treated in Phase 1, 7 in Cohort 1, and 9 in Cohort 2. No first cycle dose-limiting toxicities were seen in either cohort. Phase adverse effects (AEs) included (usually Grade 1-2) arthralgia and myalgia, peripheral neuropathy, rash, nausea, and vomiting, fatigue, alopecia, edema, dehydration, constipation, and decreased blood counts. Eleven patients completed 6 cycles of therapy. Eight patients (50%) achieved a partial response (PR). Seven of the 8 patients with evaluable samples showed increased NK cell activity when assayed 7 days after the second dose.

The carboplatin:paclitaxel:Compound (1) combination is well tolerated at the dose levels studied, and the overall safety profile appears similar to that of carboplatin:paclitaxel alone. Encouraging clinical activity was observed, as well as correlative NK activity that supports a conclusion that Compound (1) is biologically active in vivo.

The RECIST criteria used to determine objective tumor response for target lesions, taking into account the measurement of the longest diameter for all target lesions. RECIST criteria include:

Complete Response (CR): Disappearance of all target lesions

Partial Response (PR): At least a 30% decrease in the sum of the longest diameter (LD) of target lesions, taking as reference the baseline sum LD

Progressive Disease (PD): At least a 20% increase in the sum of the LD of target lesions, taking as reference the smallest sum LD recorded since the treatment started or the appearance of one or more new lesions

Stable Disease (SD): Neither sufficient shrinkage to qualify for PR nor sufficient increase to qualify for PD, talking as reference the smallest sum LD since the treatment started

Table 2 shows the substantial anticancer efficacy and NK cell activity results for different subjects. The Effector/Target data shows the ratio of the subjects PBMC cells to the NK assay target cells. The pre and post dose column values show the percent of tumor cells lysed before dosing with Paclitaxel and Compound (1). Best Response indicates an evaluation of the patient's tumor: PR=at least a 30% decrease in the sum of the longest diameters as compared to baseline, while SD indicates less than 20% of an increase and less than 30% of a decrease in the sum of the longest diameters as compared to baseline. Target Lesions indicates the percent change in targeted melanoma lesions in the subjects. NK Activity indicates the change in NK activity before and after dosing.

Table 2 shows that for patients completing the study (#1-#8) there was a substantial decrease in target lesion size for each patient. Also, 5 of the 8 patients completing the study had the best response evaluation category, at least a 30% decrease in the sum of the longest diameters compared to baseline. For NK cell activity, 8 of the 11 original patients showed an increase between pre- and post-dose treatment, though in this example the difference was not significant according to paired t-test (p=0.06).

TABLE 2

| | | % tumor cell | | | | | | |
|---|---|---|---|---|---|---|---|---|
| | | lysis | | dosing information | | | | |
| | Effector/ | pre- | post- | Paclitaxel, | Cmpnd (1) | Best | Target | NK |
| Subject | Target | dose | dose | mg/M² | mg/M² | Response | Lesions | activity |
| 1 | 80:1 | 9.55 | 16.14 | 175 | 233 | SD | −5.9% | increase |
| 2 | 80:1 | 3.12 | 8.76 | 175 | 233 | SD | −30% | increase |
| 3 | 80:1 | 7.84 | 10.05 | 175 | 233 | PR | −67% | increase |
| 4 | 80:1 | 8.4 | 5.5 | 200 | 266 | PR | −38% | decrease |
| 5 | 80:1 | 7.79 | 30.8 | 175 | 233 | PR | −34% | increase |
| 6 | 80:1 | 3.59 | 7.81 | 200 | 266 | PR | −44% | increase |
| 7 | 80:1 | 0.92 | 7.75 | 175 | 233 | SD | −24% | no change |
| 8 | 80:1 | 10.7 | 14.61 | 175 | 233 | PR | −62% | increase |
| 9 | 80:1 | 7.21 | 10.11 | | | NA | NA | increase |
| 10 | 80:1 | 8 | 3.8 | | | NA | NA | decrease |
| 11 | 80:1 | 36.19 | 45.98 | | | NA | NA | increase |

Given the safety profile of Cohort 2, this dose level (carboplatin AUC 6, paclitaxel 200 mg/m2 and Compound (1) 266 mg/m²) was used in Phase 2.

### Example 5

### A 2 Stage Phase 2 Study Shows the Disclosed Combination Therapy is Effective for Treating Advanced Metastatic Melanoma

The following study of Compound (1) and paclitaxel in patients with advanced metastatic melanoma was initiated based on the biological activity shown by the results of the above Phase I study, where the combined administration Compound (1) and paclitaxel led to dose-related Hsp70 induction.

The study included a Stage 1 initial safety assessment of the weekly dose schedule, where Compound (1) 106 mg/m2 (265 μmol/m2) and paclitaxel at 80 mg/m2 (94 μmol/m2) were administered weekly for 3 weeks out a 4 week period. The dose of Compound (1) was then escalated to 213 mg/m2 (532 μmol/m2) in combination with the paclitaxel at 80 mg/m2 (94 μmol/m2). The higher tolerated dose level was expanded to a total of 20 patients (Stage 1).

A total of 7 patients were treated in the initial safety assessment, 3 at the lower dose level and 4 at the higher. In the absence of dose-limiting toxicities in either group, the higher dose level was chosen as the dose of interest and additional patients were enrolled to complete stage 1. Adverse events seen were as expected for paclitaxel chemotherapy administration. Of 20 evaluable patients, 11 were stable at 3 months for 55% NPR.

The study will continue to Stage 2 if 7 or more patients have a response of stable disease or better, or at least 2 patients have a partial response or better. A safety assessment was performed with the first 6 patients enrolled as the weekly dose schedule had not previously been studied in humans. The primary endpoint is non-progression rate (NPR) at 3 months and response rate. Pharmacodynamic parameters include pre and post-dose NK cell activity in blood and when possible, tumor biopsies.

Table 3 shows the significant preliminary results of anticancer efficacy and NK cell activity results when assayed 7 days after the second dose for different subjects. The Effector/Target data shows the ratio of the subjects PBMC cells to the NK assay target cells. The pre and post dose column values show the percent of tumor cells lysed before dosing with Paclitaxel and Compound (1). Best Response indicates an evaluation of the patient's tumor: SD indicates less than 20% of an increase and less than 30% of a decrease in the sum of the longest diameters as compared to baseline; and PD=at least a 20% increase in the sum of the longest diameters as compared to baseline. NK Activity indicates the change in NK activity before and after dosing.

Table 3 shows that for patients completing the study (#12-#20, #22), three patients had less than 20% of an increase and less than 30% of a decrease in the sum of the longest diameters as compared to baseline, while seven patients had at least a 20% increase in the sum of the longest diameters as compared to baseline. For NK cell activity, four of the original patients showed a statistically significant increase between pre- and post-dose treatment.

TABLE 3

| | | | | dosing | | | |
|---|---|---|---|---|---|---|---|
| | | % tumor cell | | information | | Best Response | |
| | Effec- | lysis | | Pacli- | Cmpnd | cycle | |
| Sub- | tor/ | pre- | post- | taxel, | (1) | 2 | NK |
| ject | Target | dose | dose | mg/M² | mg/M² | week 4 | activity |
| 12 | 80:1 | 2.32 | 7.74 | 80 | 106 | SD | increase |
| 13 | 80:1 | 6.13 | 2.43 | 80 | 106 | PD | decrease |
| 14 | 80:1 | 3.83 | 10.77 | 80 | 213 | SD | increase |
| 15 | (40:1) | 3.5 | 10.01 | 80 | 213 | PD | (increase) |
| 16 | 80:1 | 19.71 | 19.78 | 80 | 213 | SD | no change |
| 17 | 80:1 | 41.61 | 26.52 | 80 | 213 | PD | decrease |
| 18 | 80:1 | 8.6 | 8.64 | 80 | 213 | PD | no change |
| 19 | 80:1 | 24.76 | 18.77 | 80 | 213 | PD | decrease |
| 20 | 80:1 | 16.49 | 5.2 | 80 | 213 | PD | decrease |
| 21 | 80:1 | 15.4 | 26.31 | 80 | 213 | NA | increase |
| 22 | 80:1 | 10.81 | 7.2 | 80 | 213 | PD | decrease |

The combination therapy was well-tolerated on the weekly schedule. Enrollment in the randomized portion will assess the activity of Compound (1) in combination with paclitaxel versus paclitaxel alone.

Stage 2 is planned to be a randomized 2-arm study comparing the drug combination to paclitaxel alone. The criterion for continuation to Stage 2 is >=50% non-progression rate (NPR) at two months. A total of 78 patients are to be randomized 2:1 (combination:control). The primary endpoint is time to progression; secondary endpoints are response rate, survival, and quality of life. Pharmacodynamic parameters will include pre- and post-dose measurements of NK cell activity in blood and, when possible, tumor biopsies.

### Example 6

### A Phase 2 Study Shows the Disclosed Combination Therapy is Effective for Treating Soft Tissue Sarcomas

The following study of Compound (1) and paclitaxel in patients with soft tissue sarcomas was initiated based on the biological activity shown by the results of the above Phase I study, where the combined administration Compound (1) and paclitaxel led to dose-related Hsp70 induction.

The study is a 2 stage design, enrolling 30 patients in the first stage and adding 50 patients to total 80 if certain continuation criteria are met. Major inclusion criteria are refractory or recurrent soft tissue sarcomas other than gastrointestinal stromal tumor (GIST), with evidence of recent progression. Patients are treated weekly, 3 weeks out of every 4 week cycle with 213 mg/m2 Compound (1) and 80 mg/m2 paclitaxel. For example, the compounds were administered together 3 weeks out of 4 on Days 1, 8, and 15 of a 28 day cycle as a 1 hour IV infusion. 30 Patients have been enrolled to completed accrual of Stage 1.

As used herein, “soft-tissue sarcomas” (STS) are cancers that begin in the soft tissues that support, connect, and surround various parts of the body for example, soft tissues such as muscles, fat, tendons, nerves, and blood vessels, lymph nodes, or the like. Such STSs can occur anywhere in the body, though typically about one half occur in the limbs. In various embodiments, STSs can include one or more cancers selected from liposarcoma, fibrosarcoma, malignant fibrous histiocytoma leiomyosarcoma, neurofibrosarcoma, rhabdomyosarcoma, synovial sarcoma, or the like.

Table 4 shows the significant preliminary results of anticancer efficacy and NK cell activity results when assayed 7 days after the second dose for different subjects. The Effector/Target data shows the ratio of the subjects PBMC cells to the NK assay target cells. The pre and post dose column values show the percent of tumor cells lysed before dosing with Paclitaxel and Compound (1). Best Response indicates an evaluation of the patient's tumor: PR=at least a 30% decrease in the sum of the longest diameters as compared to baseline; SD indicates less than 20% of an increase and less than 30% of a decrease in the sum of the longest diameters as compared to baseline; and PD=at least a 20% increase in the sum of the longest diameters as compared to baseline. NK Activity indicates the change in NK activity before and after dosing.

Table 4 shows that for patients completing the study (#23-#29, #31-33), five patients had less than 20% of an increase and less than 30% of a decrease in the sum of the longest diameters as compared to baseline, while five patients had at least a 20% increase in the sum of the longest diameters as compared to baseline. For NK cell activity, seven of the original patients showed a statistically significant increase or no change between pre- and post-dose treatment, while only four of the original patients showed a decrease statistically significant increase between pre- and post-dose treatment.

TABLE 4

| | | % tumor cell | | dosing information | | | |
|---|---|---|---|---|---|---|---|
| | Effec- | lysis | | Pacli- | Cmpnd | Best Response | |
| Sub- | tor/ | pre- | post- | taxel, | (1) | cycle | NK |
| ject | Target | dose | dose | mg/M² | mg/M² | 2 | activity |
| 23 | 80:1 | 4.28 | 30.48 | 80 | 213 | PD | increase |
| 24 | 80:1 | 20.74 | 20.04 | 80 | 213 | SD | no change |
| 25 | 80:1 | 34.28 | 11.86 | 80 | 213 | PD | decrease |
| 26 | 80:1 | 22.33 | 14.74 | 80 | 213 | SD | decrease |
| 27 | 80:1 | 10.6 | 22.9 | 80 | 213 | SD | increase |
| 28 | 80:1 | 17.93 | 28.13 | 80 | 213 | SD | increase |
| 29 | 80:1 | 6.58 | 17.18 | 80 | 213 | PD | increase |
| 30 | (40:1) | 9.88 | 9.91 | 80 | 213 | NA | no change |
| 31 | 80:1 | 2.62 | 5.46 | 80 | 213 | SD | increase |
| 32 | 80:1 | 13.03 | 7.41 | 80 | 213 | PD | decrease |
| 33 | 80:1 | 15.77 | 7.84 | 80 | 213 | PD | decrease |

Patients are currently being evaluated through 3 months. Adverse events seen were typical for paclitaxel administration on a similar schedule. Assessment of NK activity is ongoing. The addition of Compound (1) to the weekly paclitaxel schedule was well-tolerated, Stage 1 accrual has completed, and patients are currently being evaluated for the study continuation decision.

While this invention has been particularly shown and described with references to preferred embodiments thereof, it will be understood by those skilled in the art that various changes in form and details may be made therein without departing from the scope of the invention encompassed by the appended claims.

1. A method of increasing natural killer (NK) cell activity in a subject in need of immune system augmentation, comprising administering a bis(thio-hydrazide amide) represented by the following Structural Formula: 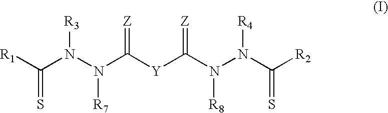
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S,
provided that the subject is not suffering from cancer, a proliferative cell disorder, a non-infective heat shock protein 70 (Hsp70) responsive disorder, or a proteasome-inhibitor responsive disorder.

2. The method of claim 1, wherein the subject is human.

3. The method of claim 2, wherein the subject has an open wound or burn injury.

4. The method of claim 2, wherein the subject has a bacterial, viral, fungal, or parasite infection, or a combination thereof.

5. The method of claim 4, wherein the subject has bacteremia.

6. The method of claim 4, wherein the subject has an intracellular infection.

7. The method of claim 4, wherein the subject has an infection caused by a fungus selected from the genera Trichophyton, Tinea, Microsporum, Epidermophyton, Aspergillus, Histoplasma, Cryptococcus, Microsporum, Candida, Malassezia, Trichosporon, Rhodotorula, Torulopsis, Blastomyces, Paracoccidioides, and Coccidioides.

8.-9. (canceled)

10. The method of claim 4, wherein the subject has an infection caused by a bacterium selected from the genera Allochromatium, Acinetobacter, Bacillus, Campylobacter, Chlamydia, Chlamydophila, Clostridium, Citrobacter, Escherichia, Enterobacter, Enterococcus, Francisella, Haemophilus, Helicobacter, Klebsiella, Listeria, Moraxella, Mycobacterium, Micrococcus, Neisseria, Proteus, Pseudomonas, Salmonella, Serratia, Shigella, Stenotrophomonas, Staphyloccocus, Streptococcus, Synechococcus, Vibrio, Yersina; Peptostreptococci, Porphyromonas, Actinomyces, Clostridium, Bacteroides, Prevotella, Anaerobiospirillum, Fusobacterium, and Bilophila.

11. (canceled)

12. The method of claim 10, wherein the subject has an intracellular bacterial infection caused by a bacterium selected from the genera Ehrlichia, Listeria; Legionella; Rickettsiae; Chlamydia; Mycobacterium; Brucella; and Coxiella.

13. The method of claim 4, wherein the subject has an infection resulting in upper respiratory tract bacterial infection, acute bacterial exacerbation of chronic bronchitis; acute community acquired pneumonia, maxillary sinus pathogenic bacteria; a urinary tract infection; or a sexually transmitted infection.

14. The method of claim 4, wherein the subject has an infection caused by a virus selected from Picornavirus; Parvoviridae; Hepatitis virus; Papovavirus; Adenovirus; Herpesvirus, Poxvirus; Calicivirus; Arbovirus; Coronavirus; a Retrovirus; Rhabdovirus; Paramyxovirus; Orthomyxovirus; Arenavirus; human T-cell Lymphotrophic virus; human papillomavirus; and human immunodeficiency virus.

15. (canceled)

16. The method of claim 4, wherein the subject has an infection caused by a parasite selected from the genera Plasmodia; Leishmania; Trypanosoma; Naegleria; Acanthamoeba; Entamoeba; Giardia lamblia; Cryptosporidium; Isospora; Cyclospora; Microsporidia; Ascaris lumbricoides; Schistosoma; Treponema; and Trichomonas.

17. (canceled)

18. The method of claim 4, wherein the subject has an infection caused by antibiotic resistant bacteria.

19. The method of claim 4, wherein the subject has an infection caused by a bacterium selected from multiple drug resistant Streptococcus pneumoniae, vancomycin resistant Enterococcus, methicillin resistant Staphylococcus Aureus, penicillin resistant Pneumococcus, antibiotic resistant Salmonella, resistant/multi-resistant Neisseria Gonorrhea, and resistant/multi-resistant Tuberculosis.

20. The method of claim 19, wherein the subject has a bacterial infection resistant to at least one antibiotic selected from penicillin, Methicillin, second generation cephalosporins, macrolides, tetracyclines, trimethoprim/methoxazole, vancomycin, tetracycline, fluoroquinolones, ceftriaxone, Cefixime, Azithromycin, Isoniazid, Rifampin, Ethambutol, Pyrazinamide, Aminoglycoside, Capreomycin, Ciprofloxacin, Ofloxacin, gemifloxacin, Cycloserine, Ethionamide, and para-aminosalicylic acid.

21. The method of claim 2, wherein the subject has an immunodeficiency disorder.

22. The method of claim 21, wherein the subject has a primary immunodeficiency disorder.

23. The method of claim 21, wherein the subject has a secondary immunodeficiency disorder.

24. The method of claim 21, wherein the subject has a disorder selected from uremia, diabetes mellitus, malnutrition, vitamin and mineral deficiencies, protein-losing enteropathies, nephrotic syndrome, myotonic dystrophy, uterine dysfunction, and sickle cell disease.

25. The method of claim 21, wherein the subject is immunosuppresed resulting from treatment with an immunosuppressive agent selected from radiation, an immunosuppressive drug, a corticosteroid, anti-lymphocyte globulin, anti-thymocyte globulin, and anti-T-cell monoclonal antibodies.

26. The method of claim 21, wherein the subject has an immunodeficiency disorder resulting from splenectomy, anesthesia, surgery, allogeneic transplant, graft-versus-host disease, or an implanted medical device.

27. The method of claim 21, wherein the subject has an immunodeficiency disorder selected from chronic fatigue syndrome, Epstein-Barr virus infection, post viral fatigue syndrome, post-transplantation syndrome, exposure to nitric oxide synthase inhibitors, aging, severe combined immunodeficiency, and variable immunodeficiency syndrome.

28. The method of claim 1, wherein the bis(thiohydrazide amide) is represented by the following structural formula: 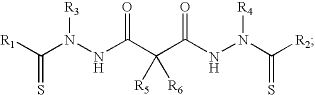
or the disodium or dipotassium salt thereof, wherein:
R₁ and R₂ are both phenyl; R₃ and R₄ are both methyl; R₅ and R₆ are both —H;
R₁ and R₂ are both phenyl; R₃ and R₄ are both ethyl; R₅ and R₆ are both —H;
R₁ and R₂ are both 4-cyanophenyl; R₃ and R₄ are both methyl; R₅ is methyl; R₆ is —H;
R₁ and R₂ are both 4-methoxyphenyl; R₃ and R₄ are both methyl; R₅ and R₆ are both —H;
R₁ and R₂ are both phenyl; R₃ and R₄ are both methyl; R₅ is methyl; R₆ is —H;
R₁ and R₂ are both phenyl; R₃ and R₄ are both ethyl; R₅ is methyl; R₆ is —H;
R₁ and R₂ are both 4-cyanophenyl; R₃ and R₄ are both methyl; R₅ and R₆ are both —H;
R₁ and R₂ are both 2,5-dimethoxyphenyl; R₃ and R₄ are both methyl; R₅ and R₆ are both —H;
R₁ and R₂ are both 2,5-dimethoxyphenyl; R₃ and R₄ are both methyl; R₅ is methyl; R₆ is —H;
R₁ and R₂ are both 3-cyanophenyl; R₃ and R₄ are both methyl; R₅ and R₆ are both —H;
R₁ and R₂ are both 3-fluorophenyl; R₃ and R₄ are both methyl; R₅ and R₆ are both —H;
R₁ and R₂ are both 4-chlorophenyl; R₃ and R₄ are both methyl; R₅ is methyl; R₆ is —H;
R₁ and R₂ are both 2-dimethoxyphenyl; R₃ and R₄ are both methyl; R₅ and R₆ are both —H;
R₁ and R₂ are both 3-methoxyphenyl; R₃ and R₄ are both methyl; R₅ and R₆ are both —H;
R₁ and R₂ are both 2,3-dimethoxyphenyl; R₃ and R₄ are both methyl; R₅ and R₆ are both —H;
R₁ and R₂ are both 2,3-dimethoxyphenyl; R₃ and R₄ are both methyl; R₅ is methyl; R₆ is —H;
R₁ and R₂ are both 2,5-difluorophenyl; R₃ and R₄ are both methyl; R₅ and R₆ are both —H;
R₁ and R₂ are both 2,5-difluorophenyl; R₃ and R₄ are both methyl; R₅ is methyl; R₆ is —H;
R₁ and R₂ are both 2,5-dichlorophenyl; R₃ and R₄ are both methyl; R₅ and R₆ are both —H;
R₁ and R₂ are both 2,5-dimethylphenyl; R₃ and R₄ are both methyl; R₅ and R₆ are both —H;
R₁ and R₂ are both 2,5-dimethoxyphenyl; R₃ and R₄ are both methyl; R₅ and R₆ are both —H;
R₁ and R₂ are both phenyl; R₃ and R₄ are both methyl; R₅ and R₆ are both —H;
R₁ and R₂ are both 2,5-dimethoxyphenyl; R₃ and R₄ are both methyl; R₅ is methyl; R₆ is —H;
R₁ and R₂ are both cyclopropyl; R₃ and R₄ are both methyl; R₅ and R₆ are both —H;
R₁ and R₂ are both cyclopropyl; R₃ and R₄ are both ethyl; R₅ and R₆ are both —H;
R₁ and R₂ are both cyclopropyl; R₃ and R₄ are both methyl; R₅ is methyl; R₆ is —H;
R₁ and R₂ are both 1-methylcyclopropyl; R₃ and R₄ are both methyl; R₅ and R₆ are both —H;
R₁ and R₂ are both 1-methylcyclopropyl; R₃ and R₄ are both methyl; R₅ is methyl and R₆ is —H;
R₁ and R₂ are both 1-methylcyclopropyl; R₃ and R₄ are both methyl; R₅ is ethyl and R₆ is —H;
R₁ and R₂ are both 1-methylcyclopropyl; R₃ and R₄ are both methyl; R₅ is n-propyl and R₆ is —H;
R₁ and R₂ are both 1-methylcyclopropyl; R₃ and R₄ are both methyl; R₅ and R₆ are both methyl;
R₁ and R₂ are both 1-methylcyclopropyl; R₃ and R₄ are both ethyl; R₅ and R₆ are both —H;
R₁ and R₂ are both 1-methylcyclopropyl; R₃ is methyl, and R₄ is ethyl; R₅ and R₆ are both —H;
R₁ and R₂ are both 2-methylcyclopropyl; R₃ and R₄ are both methyl; R₅ and R₆ are both —H;
R₁ and R₂ are both 2-phenylcyclopropyl; R₃ and R₄ are both methyl; R₅ and R₆ are both —H;
R₁ and R₂ are both 1-phenylcyclopropyl; R₃ and R₄ are both methyl; R₅ and R₆ are both —H;
R₁ and R₂ are both cyclobutyl; R₃ and R₄ are both methyl; R₅ and R₆ are both —H;
R₁ and R₂ are both cyclopentyl; R₃ and R₄ are both methyl; R₅ and R₆ are both —H;
R₁ and R₂ are both cyclohexyl; R₃ and R₄ are both methyl; R₅ and R₆ are both —H;
R₁ and R₂ are both cyclohexyl; R₃ and R₄ are both phenyl; R₅ and R₆ are both —H;
R₁ and R₂ are both methyl; R₃ and R₄ are both methyl; R₅ and R₆ are both —H;
R₁ and R₂ are both methyl; R₃ and R₄ are both t-butyl; R₅ and R₆ are both —H;
R₁ and R₂ are both methyl; R₃ and R₄ are both phenyl; R₅ and R₆ are both —H;
R₁ and R₂ are both t-butyl; R₃ and R₄ are both methyl; R₅ and R₆ are both —H;
R₁ and R₂ are ethyl; R₃ and R₄ are both methyl; R₅ and R₆ are both —H; or
R₁ and R₂ are both n-propyl; R₃ and R₄ are both methyl; R₅ and R₆ are both —H.

29. The method of claim 1, wherein the bis(thiohydrazide amide) is: 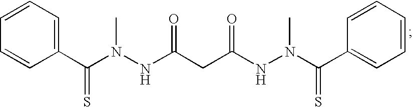
or the disodium or dipotassium salt thereof.

30. The method of claim 1, wherein the bis(thiohydrazide amide) is: 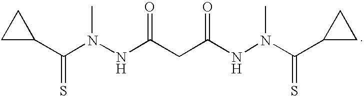
or the disodium or dipotassium salt thereof.
